(19) United States
(12) Patent Application Publication
(10) Pub. No.: US 2018/0044743 A1
(43) Pub. Date: Feb. 15, 2018

(54) DROSOPHILA MODEL OF HUMAN CERVICAL CANCER

(71) Applicants: THE CURATORS OF THE UNIVERSITY OF MISSOURI, COLUMBIA, MO (US); THE UNIVERSITY OF BRITISH COLUMBIA, VANCOUVER (CA); The Board of Regents of the University of Oklahoma, Norman, OK (US); INTERNATIONAL CENTRE FOR GENETIC ENGINEERING AND BIOTECHNOLOGY, TRIESTE (IT)

(72) Inventors: MOJGAN PADASH BARMCHI, NORMAN, OK (US); BING ZHANG, COLUMBIA, MO (US); VANESSA J. AULD, VANCOUVER (CA); LAWRENCE BANKS, TRIESTE (IT); MARY GILBERT, VANCOUVER (CA)

(21) Appl. No.: 15/673,973

(22) Filed: Aug. 10, 2017

Related U.S. Application Data
(60) Provisional application No. 62/373,007, filed on Aug. 10, 2016.

(51) Int. Cl. C12Q 1/70; G01N 33/574; C07K 14/705; C12Q 1/68

(52) U.S. Cl. CPC C12Q 1/708; C12Q 1/6886; G01N 33/574; C07K 14/705; C12Q 1/686; C12Q 1/6806; C12N 15/00

(57) ABSTRACT

Disclosed is a transgenic Drosophila model of human papillomavirus-mediated cancer. Also disclosed is a method for screening a candidate therapeutic for Human papillomavirus-induced cell transformation and a method for screening a candidate therapeutic for Human papillomavirus-induced cell transformation.

### CROSS REFERENCE TO RELATED APPLICATIONS

This application claims priority to U.S. Provisional Application No. 62/373,007 filed on Aug. 10, 2016, which is hereby incorporated by reference in its entirety.

### STATEMENT OF GOVERNMENT SUPPORT

This invention was made with government support under NIH Grant No.: RO1NS060878 awarded by the National Institutes of Health. The government has certain rights in the invention.

### INCORPORATION OF SEQUENCE LISTING

A paper copy of the Sequence Listing and a computer readable form of the Sequence Listing containing the file named “3504163_0002_ST25.txt”, which is 1,340 bytes in size (as measured in MICROSOFT WINDOWS® EXPLORER), are provided herein and are incorporated herein by reference. This Sequence Listing consists of SEQ ID NO:1-4.

### BACKGROUND OF THE DISCLOSURE

The present disclosure relates generally to human cervical cancer. More particularly, the present disclosure relates to a transgenic Drosophila model of human papillomavirus-mediated cancer. The present disclosure further relates to a method for screening a candidate therapeutic for Human papillomavirus-induced cell transformation and a method for screening a candidate therapeutic for Human papillomavirus-induced cell transformation.

Cervical cancer is the fourth leading cause of cancer death in women worldwide with ˜500,000 new cases of cervical cancer annually and ˜250,000 deaths worldwide. The main causative agents of cervical cancer are the high-risk human papillomaviruses (HPVs). HPVs can induce hyperproliferative lesions in epithelia and are responsible for >90% of cervical and anal cancers, and more than 50% of vaginal, vulvar, penile and oropharyngeal cancers as well as a significant number of head and neck squamous cell carcinomas. Although HPV vaccines are now available, it is still very important to understand the mechanism of HPV-induced tumorigenesis, given the 20 years or so lag between infection and cancer development and the low rates of vaccine uptake in many regions.

The HPV oncogenes, E6 and E7, are key to the cell transformation that underlies HPV-mediated cancer. Multiple studies have shown that E6 and E7 work cooperatively to induce carcinogenesis. E7 is critical for early stages of tumor formation, causing benign tumors and targeting Rb, whereas E6 is thought to play an important role during the later stages of tumor progression. E6 inactivates a range of targets including the tumor suppressor protein p53 and important polarity regulators including hDlg1, Scribble/Vartul and MAGI-1, all of which are directed for ubiquitin-mediated proteasomal degradation. E6 directs the degradation of many of its substrates through recruitment of an E3 ubiquitin ligase, UBE3A/E6AP, with which it forms a stable complex and redirects its activity towards the different E6 target proteins. Multiple functions of E6, including interaction with UBE3A, p53 and PDZ domain-containing substrates, appear to be required for its ability to bring about cell transformation and to contribute towards malignancy in animal models.

Mouse and human cell culture models of HPV-mediated cancer have been previously developed and have provided insight into how HPV causes cancer. However, they both suffer limitations that do not allow these models to fully understand the mechanisms of HPV-mediated cancer. Mouse models are costly and have limitations in genetic tools and techniques for manipulating genes. Similarly, cell culture models studying human cancer cell lines have not been able to provide information that can be fully translated to clinical trials and drug development. The reason for this lies in the fact that in cell culture models cells are dissociated from an in vivo environment and many aspects of the intact epithelia is absent in these models. This is highly important for understanding the mechanisms underlying tumor development and malignancy as most tumors form within an intact epithelia and the interaction between the cells surrounding the tumor and the tumor cells are very important for the cancer progression.

The availability of wide array of genetic tools and techniques in Drosophila and the high conservation of genes and signaling pathways regulating cell growth and death has made Drosophila an excellent in vivo model to study human disease including cancer. To overcome the challenges and obstacles in understanding the HPV-mediated cancer, it would be advantageous to generate a fly model of HPV-mediated cancer. The model would focus on the HPV E6 oncoprotein as it plays an essential role during cancer progression, the stage that is hardest to cure.

### BRIEF DESCRIPTION

In one aspect, the present disclosure is directed to a transgenic Drosophila co-expressing a gene encoding a human papillomavirus (HPV) E6 oncoprotein and a gene encoding human ubiquitin protein ligase E3A (UBE3A).

In one aspect, the present disclosure is directed to a method for screening a candidate therapeutic for Human papillomavirus-induced cell transformation, the method comprising: contacting the candidate therapeutic with a transgenic Drosophila, wherein the transgenic Drosophila co-expresses a gene encoding HPV E6 oncoprotein and a gene encoding human UBE3A; and analyzing the transgenic Drosophila.

In one aspect, the present disclosure is directed to a method for screening a candidate therapeutic for Human papillomavirus-induced cancer, the method comprising: contacting the candidate therapeutic with a transgenic Drosophila, wherein the transgenic Drosophila co-expresses a gene encoding HPV E6 oncoprotein and a gene encoding human UBE3A; and analyzing the transgenic Drosophila.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The disclosure will be better understood, and features, aspects and advantages other than those set forth above will become apparent when consideration is given to the following detailed description thereof. Such detailed description makes reference to the following drawings, wherein:

FIGS. 1A-1I. E6 in cooperation with UBE3A causes severe wing and eye abnormalities. Transgenes were driven with apterous-Gal4 in the wing imaginal disc. Expression of HPV18 E6 (FIG. 1A) or human UBE3A (FIG. 1B) alone had no effect on adult wing morphology. (FIG. 1C) Co-expression of E6 and UBE3A resulted in severe abnormalities. Wings are blistered and full of melanized fluid, with indistinct structure and lacking veins. Transgenes were driven with GMR-Gal4 in the eye imaginal disc. Expression of E6 (FIG. 1D) or UBE3A (FIG. 1E) alone had no effect on adult eye morphology. (FIG. 1F) Coexpression of E6 and UBE3A in the eye generated a disorganized “rough eye” phenotype. Scanning electron microscopy of eyes from (FIGS. 1D-1F). E6 (FIG. 1G) or UBE3A (FIG. 1H) alone had no effect on ommatidia organization or bristles. (FIG. 1I) When E6 and UBE3A were co-expressed, ommatidia were fused with increased bristles (arrowhead).

FIGS. 2A-2V. E6-mediated degradation of PDZ domain proteins is conserved in Drosophila. (FIGS. 2A-2P) apterous-Gal4 was used to express transgenes in the dorsal compartment of wing imaginal discs. In all images, dorsal is to the left and ventral is to the right and dashed lines indicate the boundary between the two compartments. Single Z slices are shown. E6 and UBE3A co-expression results in a severe loss of Magi (FIGS. 2A-2C), a less severe reduction of Dlg (FIGS. 2E-2G) and slightly reduced level of Scrib (FIGS. 2I-2K). Immunolabeling was quantified and the reductions were significant compared with the control dorsal side for Magi (p<0.001, n=5 discs) (FIG. 2D), Dlg (p<0.001, n=5 discs) (FIG. 2H) and Scrib (p<0.05, n=5 discs) (FIG. 2L). (FIGS. 2M-2O) Co-expression of E6 and UBE3A had no effect on the expression level or localization of P53. (FIG. 2P) Quantification of the level of P53 found no significant change (n=5 discs) (FIGS. 2Q-2S) Co-expression of an E6 transgene lacking the PDZ binding motif (E6V158A) and UBE3A did not affect the levels or localization of Magi. Quantification of the level of Magi found no significant change (n=5 discs) (FIG. 2T). (FIG. 2U) The apterous expression pattern (red) is diagrammed on the wing imaginal disc. Quantification of immunolabeling was from set standardized areas on the apterous side (black square) compared to the wild type side (white square). (FIG. 2V) Drosophila Magi and mammalian MAGI-1 are both susceptible to degradation induced by the high-risk cancer-causing HPV 16 and 18 E6 proteins. In vitro degradation assay revealed that HPV16E6 and HPV18E6 are able to degrade radiolabeled Drosophila Magi to an extent nearly equal to human MAGI. *** p<0.001, * p<0.05, ns—not statistically significant. Error bars indicate SEM. Scale bars indicate 10 μm.

FIGS. 3A-3Z′. E6 alone does not trigger degradation and E6+UBE3A does not target Baz or Ecad. UAS-transgenes were expressed under the control of apterous-Gal4 driver in the dorsal compartment of the wing imaginal discs. In all images dorsal is to the left and ventral is to the right. Dashed lines indicate the boundary between the dorsal and ventral compartments. (FIGS. 3A-3C) Co-expression of E6 and UBE3A had no effect on PDZ protein Bazooka (Baz/Par-3). (FIGS. 3E-3G) Coexpression of E6 and UBE3A had no effect on the PDZ domain protein Par-6 (FIGS. 3I-3K) Coexpression of E6 and UBE3A had no effect on the adherens junction protein Ecad. (FIGS. 3M-3O) Expression of E6 alone had no effect on the level or localization of Magi. (FIGS. 3Q-3S) Expression of E6 alone had no effect on the level or localization of Dlg. (FIGS. 3U-3W) Expression of human UBE3A alone had no effect on the levels or localization of Magi or Dlg. (FIGS. 3Y, 3Z) Co-expression of E6V158A and UBE3A had no effect on the level of Dlg (FIG. 3Y) or Scrib (FIG. 3Z). (FIGS. 3D, 3H, 3L, 3P, 3T, 3X, 3Y′, 3Z′) Graphs representing the quantification results for the levels of Baz, Ecad, Magi, Dlg, and Magi respectively. n=5 for each experiment. ns indicates that the difference is not statistically significant. Error bars indicate SEM. Scale bars indicate 10 μm.

FIG. 4. Co-expression of E6 and UBE3A in the eye disrupts the cellular integrity and structure. Expression of transgenes in the eye imaginal disc were driven with GMR-Gal4. (FIGS. 4A-4C) Expression of E6 (FIG. 4A) or UBE3A (FIG. 4B) alone had no effect on Magi. (FIG. 4C) Co-expression of E6 and UBE3A resulted in a loss of Magi. (FIGS. 4D-4F) Expression of E6 (FIG. 4D) or UBE3A (FIG. 4E) alone had no effect on Dlg. (FIG. 4F) Co-expression of E6 and UBE3A reduced the level of Dlg. (FIGS. 4G-4I) Immunolabeling with Ecad showed no effect on ommatidial organization with expression of E6 (FIG. 4G) or UBE3A alone (FIG. 4H). (FIG. 4I) Ecad immunolabeling of eyes expressing E6+UBE3A indicated disrupted cellular morphology and integrity. (FIGS. 4F and 4I) Multiple phenotypes were observed with E6+UBE3A expression including fused ommatidia observed with increased primary cone cells (yellow arrow). The number of secondary pigment cells (white arrow), primary pigment cells (purple arrowhead) and bristle cells (green arrowhead) were increased and the organization of ommatidia is also perturbed. (FIGS. 4G′-4I′) En-face view from a deeper plane of the eye at the level of the rhabdomere and photoreceptor cells. Rhabdomeres from neighboring ommatidia are fused (FIG. 4I, blue arrow) compared to FIG. 4H′ and FIG. 4G′. (FIGS. 4J-4L) Expression of E6 alone had no effect on the polarity protein Baz and junctional Armadillo, which normally localize to the zona adherens in photoreceptors and appear yellow in L and enlarged boxed area (inserts). (FIGS. 4M-4O) Co-expression of E6 and UBE3A disrupted the integrity of junctional complexes, as Baz and Arm were both mislocalized as shown by reduced amount of yellow overlap in FIG. 4O and enlarged boxed area (insets). (FIGS. 4P-4S) Diagram of an ommatidium in the eye with side views (FIGS. 4P, 4R) and corresponding cross sections (FIGS. 4Q, 4S) from the outer most level and the inner rhadomere/photoreceptor level. (FIGS. 4P, 4Q) The upper part of ommatidium is a composition of 4 cone cells (yellow) in the center of the ommatidium surrounded by two primary pigment cells (purple), 6 secondary pigment cells (beige), 3 tertiary pigment cells (red) and 3 bristle cells (green) that generate the eye bristles. FIG. 4Q represents the cells shown in FIGS. 4A-4I. (FIGS. 4R, 4S) A more interior level of the eye at the level of the eight photoreceptor cells (blue) and their rhabdomeres (gray), which are the apical domain of photoreceptors. FIG. 4S corresponds to panels FIGS. 4G′-4O. Scale bars indicate 10 μm. Insets are digitally magnified 200%.

FIG. 5. Co-expression of E6 and E6AP induces cellular transformation that is dependent on inhibition of apoptosis. All transgenes were driven with apterous-Gal4 in dorsal compartment of wing discs. In all images, dorsal is to the left and ventral is to the right and the boundary indicated by dashed lines. (FIGS. 5A-5C) Expression of E6 alone did not result in apoptosis as detected using an antibody to cleaved-caspase 3 (Cas3). (FIGS. 5D-5F) Co-expression of E6 and UBE3A causes cell death marked by the increased immunolabeling for cleaved-caspase 3 (arrow). (FIGS. 5G-5I) Co-expression of E6 and UBE3A did not increase MMP1 expression. (FIGS. 5J-5L) Blocking cell death with p35 in cells expressing E6 and UBE3A resulted in cell clusters expressing high levels of MMP1 and Baz. (FIGS. 5J′-5L′) Side projections indicating the delaminated cell clusters were on the basal side of the epithelium (yellow arrowhead). (FIGS. 5M-5O) MMP1-expressing cell clusters in E6+UBE3A+p35-expressing epithelia showed no increase in junction protein Ecad. (FIGS. 5M′-5O′) Side projection of FIGS. 5M-5O showing delaminated cell clusters on the basal side of the disk (yellow arrowhead). (FIGS. 5P-5R) Cell clusters within the apterous domain expressing E6+UBE3A+p35 had elevated MMP1 and aPKC levels. (FIGS. 5P′-5R′) Side projection of FIGS. 5P-5R showing delaminated cell clusters on the basal side of the disc strongly immunolabeled with MMP1 and aPKC (yellow arrowhead). (FIGS. 5S-5U) Dominant negative Drosophila JNK, Bsk (bsk^{DN}), blockade of JNK signaling did not suppress the cell death or elevated immunolabeling for Cas3 resulting from coexpression of E6 and UBE3A. Scale bars in FIGS. 5A-5F and FIGS. 5S-5U are 20 μm and 10 μm in all other panels.

FIG. 6. Overexpression of Drosophila Magi rescues the HPV E6+UBE3A wing phenotypes. Transgenes were expressed using apterous-Gal4 driver in the dorsal compartment of the wing disc, affecting the hinge region joining the wing to thorax and one surface of the adult wing. (FIG. 6A) Co-expression of E6 and UBE3A resulted in adult wings that were entirely blistered, full of melanized tissues and lacking wing veins. (FIG. 6B) Overexpression of Magi::Cherry with E6+UBE3A suppressed the wing blister and melanization phenotype. Wings were fully formed and all the wing veins were present. Rescue was partial with some blisters still detected. (FIGS. 6C & 6D) Overexpression of Dlg::GFP (FIG. 6C) or Scrib::GFP (FIG. 6D) in wing discs expressing E6+UBE3A did not suppress any of the E6+UBE3A-mediated phenotypes. (FIGS. 6E & 6F) Overexpression of MagiΔPDZ (FIG. 6E) did not suppress, while overexpression of MagiΔWW (FIG. 6F) in the wing discs expressing E6+UBE3A suppressed both the wing blister and melanization phenotypes. Wings were fully formed and all the wing veins were present. (FIG. 6G) The percentage of animals with rescued wings, as outlined in the Material and Methods of Example 1, were quantified (n=100 for each genotype). (FIGS. 6H-6M) Analysis of third instar wing discs. In all panels, dorsal is to the left and ventral is to the right. Dashed lines indicate the boundary between the dorsal and ventral compartments. (FIGS. 6H-6J) Overexpression of Magi (Magi::cherry) in wing discs expressing E6 and UBE3A blocked the apoptosis triggered by E6+UBE3A expression, as shown by the lack of activated Cas3 immunolabeling (Cas3). (FIGS. 6K-6M) Overexpression of Scrib::GFP in wing discs expressing E6+UBE3A did not block apoptosis, as shown by activated Cas3 immunolabeling (arrow). Scale bar indicates 10 μm.

FIG. 7. E6 targets the PDZ domains of Magi. Transgenes were expressed in the wing disc under the control of apterous-Gal4 driver in the dorsal compartment. Discs are oriented in all panels with dorsal to the left and ventral to the right. (FIGS. 7A & B) Full length Magi::Cherry was distributed around the membrane and found in prominent intracellular puncta (FIGS. 7A & 7A′). When co-expressed with E6+UBE3A, Magi::Cherry levels in the membrane and puncta were reduced (FIGS. 7B & 7B′) with regions of little or no expression (FIG. 7B′ arrow). (FIGS. 7C & 7D) MagiΔWW is uniformly distributed around the membrane (FIGS. 7C & 7C′). When expressed with E6+UBE3A (FIGS. 7D & 7D′) the levels of MagiΔWW are reduced with regions of little or no expression (FIG. 7D′ arrow). (FIGS. 7E & 7F) The localization and levels of MagiΔPDZ were not affected in E6+UBE3A expressing compartment (FIGS. 7F & 7F′) similar to MagiΔPDZ expression alone (FIGS. 7E & 7E′). (FIG. 7G) Cartoon of the Magi transgenic constructs with the WW (yellow), PDZ (blue) and epitope tags (Cherry-red; FLAG-black) indicated. Scale bars indicate 10 μm. Inserts were digitally magnified 200%.

FIG. 8. HPV E6 in conjunction with oncogenic Ras and Notch causes tumorigenesis and malignancy. (FIGS. 8A-8H) Transgenes were expressed under the control of apterous-Gal4 in the dorsal compartment of the wing disc. Dorsal is up and ventral is down in these panels. Dashed lines indicate the boundary between the dorsal and ventral compartments. (FIGS. 8A-8D) Expression of constitutively activated Ras with E6+UBE3A for 24 hours in wing epithelia. (FIGS. 8A & 8B) Cells positive for E6::myc displayed a flat and fibroblast-like morphology (arrowheads). (FIG. 8B) A higher magnification of FIG. 8A shows the expression is greater in single or small cell clusters. (FIG. 8C) Digital magnification of FIG. 8B shows cells displaying filopodial-like processes (arrow). (FIG. 8D) At 24 hours of expression, E6::Myc (red) labeled cells were found on the basal side of the epithelium (arrow), with the remaining Magi immunolabeling (green) marking the apical side. (FIG. 8E) Expression of constitutively activated Ras85DV12 (RasV12). Over proliferation and the generation of extra folds in the tissues was observed but no mesenchymal-like cells or clusters of MMP1 expressing cells were seen. (FIGS. 8F-8H) Expression of Ras85DV12 with E6+UBE3A for 48 hours. (FIG. 8F) Both individual and cell clusters that expressed high levels of MMP1 were observed and were limited to the apterous side. (FIG. 8G) Higher magnification of panel FIG. 8F showing the filopodial-like processes observed with many clusters (arrow). (FIG. 8H) A side projection of E6+UBE3A plus Ras85DV12 expressing disc. The MMP1 positive cell clusters are on the basal side of the epithelium (arrowhead). (FIGS. 8I-8N) Transgenes were expressed with the eye-specific driver, GMR-Gal4, and labeled with a membrane tagged GFP. Pupae (FIGS. 8I, 8J and 8M) or pharate adults (FIGS. 8K, 6L and 8N) are shown with anterior to the right and posterior to the left. (FIG. 8I) Activated Ras and GFP-positive cells do not migrate out of the developing eyes (arrowhead). (FIG. 8J) Expression of E6+UBE3A in the presence of Ras85DV12 resulted in migration, with many GFP-positive cells detected in the abdomen of these animals (arrow), distant from the source of E6+UBE3A+Ras85DV12 expression in the eye imaginal disc (arrowhead). (FIG. 8K) Activated Notch, NotchACT, and GFP positive cells do not migrate out of the developing eyes (arrowhead). (FIG. 8L) When E6+UBE3A were coexpressed with NotchACT, cells labeled with GFP were observed in the abdomen (arrow), far from the source of the expression (arrowhead). (FIG. 8M) Co-expression of E6V158A and UBE3A resulted reduced cell migration in the abdomen (arrow) away from the eye disc (arrowhead), compared with wildtype E6. (FIG. 8N) When E6V158A+UBE3A were co-expressed with Notch ACT, no GFP-labeled cells were observed in the abdomen away from the eye disc (arrowhead). Scale bars: FIG. 8A 20 μm, FIGS. 8B-8H 10 μm, FIGS. 8I-8N 100 μm.

FIG. 9. E6+UBE3A-mediated defects are not mediated by other targets of E6 including regulators of apoptotic cell death and P53. Transgenes were driven with eye specific GMR-Gal4 driver. (FIG. 9A) E6+UBE3A expressing eye with a rough eye phenotype. Reduction of Buffy (FIG. 9B), Debcl (FIG. 9C) or overexpression of Debcl (FIG. 9D) had no effect on the E6+UBE3A mediated eye defects. (FIG. 9E) Expression of a dominant negative form of P53 (P53 H159N) had no effect on the E6+UBE3A eye phenotype. (FIG. 9F) Control eye for D in which overexpression of Debcl has a similar effect on the eye as when Debcl is co-expressed with E6+UBE3A.

FIG. 10. A signaling pathway screen identified the insulin receptor as an E6 modifier. Transgenes were driven with eye specific GMR-Gal4 driver. (FIG. 10A) Wild type eye. (FIG. 10B) E6+UBE3A expressing eye with a rough eye phenotype. (FIG. 10C) A dominant negative Insulin Receptor (InRDN) alone with eyes smaller than wild type. (FIG. 10D) A dominant negative InR (InRDN) together with E6+UBE3A caused necrosis (black regions). (FIG. 10E) Insulin receptor (InR) overexpression alone increased eye size. (FIG. 10F) Insulin Receptor (InR) together with E6+UBE3A triggered hyperplasia without-pocketing of eye tissue. (FIG. 10G) Activated MAPK (Erk, Drosophila rolled) caused a rough eye. (FIG. 10H) Activated rolled, expressed simultaneously with E6+UBE3A, had no additional effect on the E6+UBE3A phenotypes. (FIG. 10I) A dominant negative form of EGFR (EGFR DN) alone caused very small eye phenotype. (FIG. 10J) Dominant negative EGFR (EGFR DN) together with E6+UBE3A had no additional effect on E6+UBE3A phenotypes. (FIG. 10K) Activated beta-catenin (Drosophila armadillo (armS10)) resulted in small eye phenotype. (FIG. 10L) Activated arm (armS10) together with E6+UBE3A, had no additional effect on E6+UBE3A. (FIG. 10M) The downstream effector of Hippo signaling, Yki::GFP had no eye phenotype. (FIG. 10N) Yki::GFP together with E6+UBE3A neither enhanced or suppressed the E6+UBE3A phenotype. (FIG. 10O) A dominant negative JNK, (Drosophila Bsk DN) together with E6+UBE3A had no additional effect on the E6+UBE3A eye phenotype. (FIG. 10P) Two copies of the E6 transgene plus UBE3A enhanced the defects caused by E6+UBE3A expression. Eyes are smaller and glossy compared to eyes with one copy of E6 plus UBE3A (compare with FIG. 10B).

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

Drosophila melanogaster has been widely and successfully used as a powerful genetic model organism to study human diseases, including cancer, owing to the strong conservation of genes and signaling pathways between Drosophila and human. Many tumor suppressor genes, including Dlg, Scribble, and Lgl, as well as oncogenic signaling pathways such as Notch, were first identified in Drosophila. Many epithelial derived tumors have been modeled in flies. For example, oncogenic Ras or Notch paired with loss of function mutations in Scribble result in the formation of metastatic tumors in Drosophila that share many characteristics with human tumors.

In one aspect, the present disclosure is directed to a transgenic Drosophila co-expressing a gene encoding a human papillomavirus (HPV) E6 oncoprotein and a gene encoding human ubiquitin protein ligase E3A (UBE3A).

In one embodiment, the transgenic Drosophila further expresses a gene encoding a Drosophila insulin receptor.

Suitably, co-expression of the gene encoding HPV E6 oncoprotein and the gene encoding human UBE3A is in a tissue selected from eye, wing, and combinations thereof. Suitably, co-expression of the gene encoding HPV E6 oncoprotein and the gene encoding human UBE3A is in a tissue selected from the group consisting of eye, wing, and combinations thereof.

Tissue co-expressing a gene encoding a human papillomavirus (HPV) E6 oncoprotein and a gene encoding human ubiquitin protein ligase E3A (UBE3A) exhibits a cellular abnormality. Particular cellular abnormalities that the tissue can express include a small wing size phenotype, a blistered wing phenotype, melanized wing epithelia, rough eye phenotype, glossy eye phenotype, disorganized ommatidia, fused ommatidia, an increase in cone cell number, an increase in primary pigment cell number, an increase in secondary pigment cell number, an increase in tertiary pigment cell number, an increase in bristle cell number, an increase in bristle number, mislocalization of a polarity marker protein, and combinations thereof.

Suitably, co-expression of the gene encoding HPV E6 oncoprotein and the gene encoding human UBE3A reduces a Drosophila homolog of a human PDZ protein. The Drosophila homolog of a human PDZ protein is selected from Membrane Associated Guanylate Kinase, WW And PDZ Domain Containing 1 (Magi), discs large homolog 1 scribble cell polarity complex component (Dlg), Scribbled Planar Cell Polarity Protein (Scribble), and combinations thereof. The Drosophila homolog of a human PDZ protein is selected from the group consisting of Membrane Associated Guanylate Kinase, WW And PDZ Domain Containing 1 (Magi), discs large homolog 1 scribble cell polarity complex component (Dlg), Scribbled Planar Cell Polarity Protein (Scribble), and combinations thereof.

Suitably, the HPV E6 oncoprotein includes a PDZ binding motif. The HPV E6 oncoprotein is selected from HPV 16 E6, HPV 18 E6, HPV 31 E6, HPV 33 E6, HPV 35 E6, HPV 39 E6, HPV 45 E6, HPV 51 E6, HPV 52 E6, HPV 56 E6, HPV 58 E6, HPV 59 E6, and combinations thereof. The HPV E6 oncoprotein is selected from the group consisting of HPV 16 E6, HPV 18 E6, HPV 31 E6, HPV 33 E6, HPV 35 E6, HPV 39 E6, HPV 45 E6, HPV 51 E6, HPV 52 E6, HPV 56 E6, HPV 58 E6, HPV 59 E6, and combinations thereof.

In another aspect, the present disclosure is directed to a method for screening a candidate therapeutic for Human papillomavirus-induced cell transformation. The method includes: contacting the candidate therapeutic with a transgenic Drosophila, wherein the transgenic Drosophila co-expresses a gene encoding HPV E6 oncoprotein and a gene encoding human UBE3A; and analyzing the transgenic Drosophila.

Suitably, co-expression of the gene encoding HPV E6 oncoprotein and the gene encoding human UBE3A is in a tissue selected from eye, wing, and combinations thereof. Suitably, co-expression of the gene encoding HPV E6 oncoprotein and the gene encoding human UBE3A is in a tissue selected from the group consisting of eye, wing, and combinations thereof.

Suitably, co-expression of the gene encoding HPV E6 oncoprotein and the gene encoding human UBE3A results in a cellular abnormality. The cellular abnormality comprises a small wing size phenotype, a blistered wing phenotype, melanized wing epithelia, rough eye phenotype, glossy eye phenotype, disorganized ommatidia, fused ommatidia, an increase in cone cell number, an increase in primary pigment cell number, an increase in secondary pigment cell number, an increase in tertiary pigment cell number, an increase in bristle cell number, an increase in bristle number, mislocalization of a polarity marker protein, and combinations thereof.

Suitably, co-expression of the gene encoding HPV E6 oncoprotein and the gene encoding human UBE3A reduces a Drosophila homolog of a human PDZ protein. The Drosophila homolog of a human PDZ protein is selected from Membrane Associated Guanylate Kinase, WW And PDZ Domain Containing 1 (Magi), discs large homolog 1 scribble cell polarity complex component (Dlg), Scribbled Planar Cell Polarity Protein (Scribble), and combinations thereof. The Drosophila homolog of a human PDZ protein is selected from the group consisting of Membrane Associated Guanylate Kinase, WW And PDZ Domain Containing 1 (Magi), discs large homolog 1 scribble cell polarity complex component (Dlg), Scribbled Planar Cell Polarity Protein (Scribble), and combinations thereof.

The transgenic Drosophila can further express a gene encoding a Drosophila insulin receptor.

The transgenic Drosophila that co-expresses the gene encoding HPV E6 oncoprotein and the gene encoding human UBE3A, and further co-expresses the gene encoding the Drosophila insulin receptor can be in a tissue selected from eye, wing, and combinations thereof. The transgenic Drosophila that co-expresses the gene encoding HPV E6 oncoprotein and the gene encoding human UBE3A, and further co-expresses the gene encoding the Drosophila insulin receptor can be in a tissue selected from the group consisting of eye, wing, and combinations thereof.

The transgenic Drosophila that co-expresses the gene encoding HPV E6 oncoprotein, the gene encoding human UBE3A, and further co-expresses the gene encoding the Drosophila insulin receptor results in excessive overgrowth.

Suitably, the HPV E6 oncoprotein is selected from HPV 16 E6, HPV 18 E6, HPV 31 E6, HPV 33 E6, HPV 35 E6, HPV 39 E6, HPV 45 E6, HPV 51 E6, HPV 52 E6, HPV 56 E6, HPV 58 E6, HPV 59 E6, and combinations thereof. Suitably, the HPV E6 oncoprotein is selected from the group consisting of HPV 16 E6, HPV 18 E6, HPV 31 E6, HPV 33 E6, HPV 35 E6, HPV 39 E6, HPV 45 E6, HPV 51 E6, HPV 52 E6, HPV 56 E6, HPV 58 E6, HPV 59 E6, and combinations thereof.

In another aspect, the present disclosure is directed to a method for screening a candidate therapeutic for Human papillomavirus-induced cancer. The method include: contacting the candidate therapeutic with a transgenic Drosophila, wherein the transgenic Drosophila co-expresses a gene encoding HPV E6 oncoprotein and a gene encoding human UBE3A; and analyzing the transgenic Drosophila.

In one embodiment, the transgenic Drosophila further expresses a gene encoding a Drosophila insulin receptor.

Suitably, co-expression of the gene encoding HPV E6 oncoprotein and the gene encoding human UBE3A is in a tissue selected from eye, wing, and combinations thereof. Suitably, co-expression of the gene encoding HPV E6 oncoprotein and the gene encoding human UBE3A is in a tissue selected from the group consisting of eye, wing, and combinations thereof.

Tissue co-expressing a gene encoding a human papillomavirus (HPV) E6 oncoprotein and a gene encoding human ubiquitin protein ligase E3A (UBE3A) exhibits a cellular abnormality. Particular cellular abnormalities that the tissue can express include a small wing size phenotype, a blistered wing phenotype, melanized wing epithelia, rough eye phenotype, glossy eye phenotype, disorganized ommatidia, fused ommatidia, an increase in cone cell number, an increase in primary pigment cell number, an increase in secondary pigment cell number, an increase in tertiary pigment cell number, an increase in bristle cell number, an increase in bristle number, mislocalization of a polarity marker protein, and combinations thereof.

Suitably, co-expression of the gene encoding HPV E6 oncoprotein and the gene encoding human UBE3A reduces a Drosophila homolog of a human PDZ protein. The Drosophila homolog of a human PDZ protein is selected from Membrane Associated Guanylate Kinase, WW And PDZ Domain Containing 1 (Magi), discs large homolog 1 scribble cell polarity complex component (Dlg), Scribbled Planar Cell Polarity Protein (Scribble), and combinations thereof. The Drosophila homolog of a human PDZ protein is selected from the group consisting of Membrane Associated Guanylate Kinase, WW And PDZ Domain Containing 1 (Magi), discs large homolog 1 scribble cell polarity complex component (Dlg), Scribbled Planar Cell Polarity Protein (Scribble), and combinations thereof.

Suitably, the HPV E6 oncoprotein includes a PDZ binding motif. The HPV E6 oncoprotein is selected from HPV 16 E6, HPV 18 E6, HPV 31 E6, HPV 33 E6, HPV 35 E6, HPV 39 E6, HPV 45 E6, HPV 51 E6, HPV 52 E6, HPV 56 E6, HPV 58 E6, HPV 59 E6, and combinations thereof. The HPV E6 oncoprotein is selected from the group consisting of HPV 16 E6, HPV 18 E6, HPV 31 E6, HPV 33 E6, HPV 35 E6, HPV 39 E6, HPV 45 E6, HPV 51 E6, HPV 52 E6, HPV 56 E6, HPV 58 E6, HPV 59 E6, and combinations thereof.

In another aspect, the present disclosure is directed to a method for screening a candidate therapeutic for Human papillomavirus-induced cell transformation. The method includes: contacting the candidate therapeutic with a transgenic Drosophila, wherein the transgenic Drosophila co-expresses a gene encoding HPV E6 oncoprotein and a gene encoding human UBE3A; and analyzing the transgenic Drosophila.

Suitably, co-expression of the gene encoding HPV E6 oncoprotein and the gene encoding human UBE3A is in a tissue selected from eye, wing, and combinations thereof. Suitably, co-expression of the gene encoding HPV E6 oncoprotein and the gene encoding human UBE3A is in a tissue selected from the group consisting of eye, wing, and combinations thereof.

Suitably, co-expression of the gene encoding HPV E6 oncoprotein and the gene encoding human UBE3A results in a cellular abnormality. The cellular abnormality comprises a small wing size phenotype, a blistered wing phenotype, melanized wing epithelia, rough eye phenotype, glossy eye phenotype, disorganized ommatidia, fused ommatidia, an increase in cone cell number, an increase in primary pigment cell number, an increase in secondary pigment cell number, an increase in tertiary pigment cell number, an increase in bristle cell number, an increase in bristle number, mislocalization of a polarity marker protein, and combinations thereof.

Suitably, co-expression of the gene encoding HPV E6 oncoprotein and the gene encoding human UBE3A reduces a Drosophila homolog of a human PDZ protein. The Drosophila homolog of a human PDZ protein is selected from Membrane Associated Guanylate Kinase, WW And PDZ Domain Containing 1 (Magi), discs large homolog 1 scribble cell polarity complex component (Dlg), Scribbled Planar Cell Polarity Protein (Scribble), and combinations thereof. The Drosophila homolog of a human PDZ protein is selected from the group consisting of Membrane Associated Guanylate Kinase, WW And PDZ Domain Containing 1 (Magi), discs large homolog 1 scribble cell polarity complex component (Dlg), Scribbled Planar Cell Polarity Protein (Scribble), and combinations thereof.

The transgenic Drosophila can further express a gene encoding a Drosophila insulin receptor.

The transgenic Drosophila that co-expresses the gene encoding HPV E6 oncoprotein and the gene encoding human UBE3A, and further co-expresses the gene encoding the Drosophila insulin receptor can be in a tissue selected from eye, wing, and combinations thereof. The transgenic Drosophila that co-expresses the gene encoding HPV E6 oncoprotein and the gene encoding human UBE3A, and further co-expresses the gene encoding the Drosophila insulin receptor can be in a tissue selected from the group consisting of eye, wing, and combinations thereof.

The transgenic Drosophila that co-expresses the gene encoding HPV E6 oncoprotein, the gene encoding human UBE3A, and further co-expresses the gene encoding the Drosophila insulin receptor results in excessive overgrowth.

Suitably, the HPV E6 oncoprotein is selected from HPV 16 E6, HPV 18 E6, HPV 31 E6, HPV 33 E6, HPV 35 E6, HPV 39 E6, HPV 45 E6, HPV 51 E6, HPV 52 E6, HPV 56 E6, HPV 58 E6, HPV 59 E6, and combinations thereof. Suitably, the HPV E6 oncoprotein is selected from the group consisting of HPV 16 E6, HPV 18 E6, HPV 31 E6, HPV 33 E6, HPV 35 E6, HPV 39 E6, HPV 45 E6, HPV 51 E6, HPV 52 E6, HPV 56 E6, HPV 58 E6, HPV 59 E6, and combinations thereof.

Establishing a Fly Model of HPV E6 and Human UBE3A

In order to establish a fly model of HPV-mediated cellular abnormalities a construct was first generated that carried the sequence of the human HPV 18E6 tagged with Myc downstream of a UAS sequence that is a binding site for the transcription activator GAL4. This construct was injected into fly embryos to generate transgenic flies carrying the UAS-HPV18E6-Myc in their genome. UAS-HPV18E6-Myc flies were crossed to another fly that carried GAL4 which were only expressed in the eye or wing, GMR-GAL4 and Apterous-GAL4 respectively (GAL4 lines are all available through Drosophila stock centers). In this way the expression of HPV18E6 is only driven in the eye or wing where the Gal4 is expressed as the GAL4 is the only activator of the UAS-HPV18E6 expression. This technique, GAL4-UAS expression, is a standard technique that is frequently used in Drosophila research to drive the expression of genes in tissue and cell specific manner. Expressing HPV18E6 alone in the wing or eye epithelia, using the wing-specific driver (apterous) and eye-specific driver (GMR), respectively, was not sufficient to induce any abnormalities within these tissues.

E6 requires the human E3 ubiquitin ligase (UBE3A/E6AP) for degradation of many of its substrates and while Drosophila has a UBE3A homologue, it was possible that HPV18 E6 was unable to activate or interact with the Drosophila UBE3A. In support of this we found that expression of E6 together with a previously established transgene that expresses human UBE3A (Reiter L. T et al., 2006), resulted in severe abnormalities in wing and eye epithelia. These abnormalities included smaller and blistered wings that were full of melanized tissue and were held out. Similarly, co-expression of E6 and human UBE3A in the eye resulted in rough eyes with disorganized and fused ommatidia as well as increase in number of bristles. These abnormalities were specific to co-expression of E6 and UBE3A and were 100% penetrant, whilst neither E6 nor UBE3A expression alone resulted in any defects. These results show that expression of E6+UBE3A is deleterious in Drosophila epithelia, indicating conservation of cellular targets downstream of the E6+UBE3A complex.

The mechanism of E6-mediated cellular defects is conserved between Drosophila and human. The wing and eye phenotype indicates that similar to its role in vertebrate epithelial cells E6 requires UBE3A to exert its cellular effects in Drosophila epithelial cells. These results prompted us to ask whether any of the known targets of E6 in cancer cells are also targeted in Drosophila epithelial cells. E6 targets PDZ domain proteins of the polarity and junctional network including MAGI-1, hDlg1 and Scribble/Vartul for ubiquitin-mediated proteasomal degradation. The level and localization of these targets in both wing and eye epithelia were analyzed. E6+UBE3A co-expression led to a significant loss of Magi from the adherens junction domain, a modest reduction in the levels of Dlg, and a weak reduction in the levels of Scrib. These targets were specific for E6 as the levels and localization of other PDZ proteins such as Bazooka (Drosophila Par-3) and Par-6 did not show any changes. Similarly, there were no changes in the level or localization of E-cadherin at the adherens junction. These results indicate that E6 binding and degradation of select PDZ proteins is specific.

Expression of E6 or UBE3A alone did not result in any changes in the levels or localization of Magi, Dlg or Scribble confirming that alterations to the PDZ proteins are a result of the E6 and UBE3A complex Similar results were obtained when E6+UBE3A is expressed in the eye epithelia using an eye specific driver. These results are consistent with previous results from human cervical cancer cells in which MAGI-1, hDlg-1 and hScrib are targeted for degradation by E6.

These results indicate that Drosophila Magi is particularly susceptible to E6 targeting, and is consistent with studies performed in mammalian cells, where MAGI-1 is one of the most strongly bound E6 PDZ targets and is also very efficiently degraded. These observations indicate that the mechanism of E6 targeting these PDZ cell polarity-regulating proteins is conserved between humans and Drosophila.

As Magi appears to be a major target of HPV E6+UBE3A in Drosophila the ability of E6 to degrade human MAGI-1 and Drosophila Magi was compared in vitro. Human E6 was degraded by Drosophila Magi in human cells. Drosophila Magi and mammalian MAGI-1 are equivalently susceptible to degradation induced by the high-risk cancer-causing HPV 16 and 18 E6 proteins. These findings highlight the high degree of evolutionary conservation in this particular HPV E6 target.

HPV E6-mediated degradation of PDZ proteins in human epithelial cells uses an intact PDZ-binding motif (PBM) at the C-terminus of the E6 protein. Whether the loss or reduction of PDZ proteins in the Drosophila epithelial cells was also dependent upon an intact E6 PBM was analyzed. A UAS-HPV18E6V158A transgene tagged with HA was generated. This transgene carries a point mutation that disrupts the E6 PBM (E6V158A) and prevents E6 binding to PDZ domain proteins. When co-expressed with UBE3A, the E6V158A mutant did not trigger a loss of Magi, Dlg or Scrib. These results confirm that E6 targeting of Drosophila Magi also uses an intact E6 PBM.

HPV E6 in conjunction with oncogenic Ras and Notch causes tumorigenesis and malignancy. In humans there is usually a long latency for cancer development in presence of HPV infection, indicating that cooperation between E6 and UBE3A is insufficient to cause uncontrolled growth and malignant transformation of epithelia. Genetic events, such as genomic instability and spontaneous mutation have been shown to play a role in E6 induced epithelial transformation. The results are consistent with this view as coexpression of E6+UBE3A was not sufficient to cause cellular transformation and cancer. Mutations in oncogenes such as Ras have been implicated in cancer progression and HPV-mediated tumorigenesis.

To investigate this in the model system of the present disclosure, E6+UBE3A was expressed in epithelial cells that expressed an activated Ras, Ras85DV12. In the presence of a constitutive active Ras, some epithelial cells that expressed high levels of E6 had altered morphology. These cells morphologically resembled mesenchymal cells in that they acquired a fibroblast-like, flat morphology, displayed filopodial-like processes and were delaminated at the basal side of the columnar epithelia and expressed MMP1.

Overexpression of Ras85DV12 in the absence of E6+UBE3A caused disc overgrowth but no mesenchymal-like cells or MMP1 cell clusters. These results indicate that the combination of HPV E6 and UBE3A is insufficient to cause cellular transformation, but requires the cooperation of cellular oncogenes, such as activated Ras. Only a small proportion of E6 expressing cells underwent transformation, as initially altered morphologies were only seen in single cells, which over time expanded into clusters in a manner analogous to the clonal development of HPV-induced malignancies.

HPV E6 has been implicated in the later stages of tumorigenesis. Therefore, whether coexpression of E6+UBE3A and Ras85DV12 could cause malignancy and metastasis was analyzed. To do this E6+UBE3A, Ras85DV12 and membrane-bound mCD8::GFP were expressed using an eye-specific Gal4 driver to detect the migration of malignant cells into other regions of the body. Expression of E6+UBE3A in the presence of Ras85DV12 resulted in cellular migration with many GFP positive cells detected in the abdomen of these animals, distant from the source of expression in the eye imaginal disc.

Similar results were obtained when E6+UBE3A were co-expressed with an activated form of Notch (NotchACT) where E6+UBE3A+NotchAct was lethal at the pharate adult stage (prior to hatching). NotchACT plus E6+UBE3A cells labeled with GFP were observed in the abdomen distant to the eye imaginal disc. These results indicate that E6 plus UBE3A when expressed with activated Ras or Notch leads to many of the cellular phenotypes associated with EMT.

Magi and insulin receptor as two modifiers of E6-mediated cellular transformation. As Magi was strongly reduced by E6+UBE3A expression, whether Magi overexpression could suppress and rescue the defects caused by E6+UBE3A in the wing epithelia was analyzed. Co-overexpression of a cherry tagged form of Drosophila Magi (Magi::Cherry) and E6+UBE3A partially rescued the adult wing phenotypes. This rescue was dependent on the PDZ domains of Magi and independent of the WW domains. The ability of Magi to rescue the E6+UBE3A phenotypes was specific as the other PDZ proteins such as Dlg and Scrib targeted by E6 failed to rescue E6+UBE3A-mediated wing abnormalities.

Given the high degree of conservation of its genes and signaling pathways the Drosophila eye appeared to be an ideal model system to identify the signaling pathways involved in E6+UBE3A mediated cell abnormalities. Therefore, a small genetic screen was used to identify which signaling molecules and pathways functionally interact with E6. A range of signaling pathways were analyzed and observed no enhancement or suppression of the E6+UBE3A phenotypes with the exception of the insulin receptor. When the insulin signaling was blocked by expressing a dominant negative form of the insulin receptor (InRDN) in conjunction with E6+UBE3A, the resulting eyes displayed large necrotic scars, which is an indication of cell death. Co-expression of the insulin receptor (InR) in E6+UBE3A expressing eyes resulted in hyperplasia compared with the expression of insulin receptor alone, which generated bigger eyes but no hyperplasia. In contrast, changes to the EGF receptor (EGFR), JNK (Drosophila Bsk), Wnt signaling with activated beta catenin (Drosophila Arm) or Hippo signaling (Drosophila Yki) had no effect on the E6+UBE3A eye phenotypes. Thus, none of the other pathways tested showed any effect upon the E6+UBE3A phenotype; indicating that the effect of the insulin receptor is specific and that changes in insulin signaling may play an important role in the cell transformation and cancer progression induced by HPV.

The model of the present disclosure is the first fly model of HPV-mediated disease and serves as an in vivo model that can be used as a platform to discover novel therapeutics for HPV-mediated cancer. Additionally due to power of fly genetics and availability of sophisticated genetic tools and techniques this model is a unique and invaluable in vivo model to understand the molecular mechanism underlying HPV-mediated cellular abnormalities.

Relative to mouse models the fly model provides a rapid, ease and very cost effective whole genome genetic screen as well as small chemicals/compounds screening to discover novel proteins and therapeutic molecules that can affect or ameliorate E6-induced cellular transformation. Such screens would be impossible in mouse models of HPV as it is extremely expensive, tedious and labor-intensive. Cell culture models of human cancer cells are not an ideal system for such screens either, as they do not represent the actual condition in the living animal due to dissociation of cells from the body. Hence the HPV E6 fly model provides an in vivo model allowing for whole genome genetic screening and drug screening.

Various functions and advantages of these and other embodiments of the present disclosure will be more fully understood from the examples shown below. The examples are intended to illustrate the benefits of the present disclosure, but do not exemplify the full scope of the disclosure.

### Example 1

In this Example, an in vivo model of HPV E6-mediated cellular transformation using Drosophila was developed.

### Material and Methods

Fly Strains and Genetics

The following Drosophila strains were used, UAS-Magi::Cherry (Padash Barmchi M, Samarasekera G, Gilbert M, Auld V J, Zhang B (2016) Magi Is Associated with the Par Complex and Functions Antagonistically with Bazooka to Regulate the Apical Polarity Complex. PLoS One 11: e0153259), UAS-Dlg::GFP (Koh Y H, Popova E, Thomas U, Griffith L C, Budnik V (1999) Regulation of DLG localization at synapses by CaMKII-dependent phosphorylation. Cell 98: 353-363), UAS-MagiΔPDZ and UAS-MagiΔWW (Beller M, Blanke S, Brentrup D, Jackle H (2002) Identification and expression of Ima, a novel Ral-interacting Drosophila protein. Mech Dev 119 Suppl 1: S253-260), UAS-hUBE3A (Reiter L T, Seagroves T N, Bowers M, Bier E (2006) Expression of the Rho-GEF Pbl/ECT2 is regulated by the UBE3A E3 ubiquitin ligase. Hum Mol Genet 15: 2825-2835), Scrib::GFP (Buszczak M, Paterno S, Lighthouse D, Bachman J, Planck J, Owen S, et al. (2007) The carnegie protein trap library: a versatile tool for Drosophila developmental studies. Genetics 175: 1505-1531), UAS-Scrib::GFP (Zeitler J, Hsu C P, Dionne H, Bilder D (2004) Domains controlling cell polarity and proliferation in the Drosophila tumor suppressor Scribble. J Cell Biol 167: 1137-1146). UAS-p35, UAS-mCD8::GFP, UAS-InR, UAS-InR DN, UAS-ArmS10, EGFR DN, UAS-rl^{Sem}, UAS-Yki::GFP, UAS-Notch Intra, UAS-Bsk DN, UAS-Ras85D.V12, P53::GFP, UAS-P53 H159N, UAS-Debcl, buffy^{H37}, Debcl^{E26}, apterous-Gal4, GMR-Gal4, Gal80^{ts} and all the transgenes and mutants used in Table 1 were from the Bloomington Drosophila Stock Center.

TABLE 1
Signaling pathway screen to identify
E6 + UBE3A interacting genes

| Genes tested | Allele type | Signaling Pathway | Effect on |
|---|---|---|---|
| UAS-InR | Gain of function | Insulin signaling | Enhanced |
| UAS-InR DN | Loss of function | Insulin signaling | Enhanced |
| | | | necrosis |
| Akt[04226]/+ | Loss of function | PI3K signaling | No effect |
| | Strong hypomorph | | |
| UAS-Rolled Act | Gain of function | MAPK signaling | No effect |
| UAS-EGFR DN | Loss of function | EGFR signaling | No effect |
| UAS-Ras85D DN | Loss of function | Ras signaling | No effect |
| UAS-Arm S10 | Gain of function | Wnt signaling | No effect |
| UAS-Yki::GFP | Gain of function | Hippo signaling | No effect |
| hh[AC]/+ | Loss of function | Hedgehog | No effect |
| | amorphic | | |
| wit[A12]/+ | Loss of function | BMP signaling | No effect |
| | Strong hypomorph | | |
| UAS-BskDN | Loss of function | JNK signaling | No effect |
| UAS-Dronc DN | Loss of function | Apoptosis - JNK | No effect |
| Eiger-RNAi | Loss of function | Apoptosis - JNK | No effect |
| Tak1[2527]/+ | Loss of function | JNK signaling | No effect |
| buffy[H37]/+ | Loss of function | Apoptosis | No effect |
| DebclE26[ ]/+ | Loss of function | Apoptosis | No effect |
| UAS-Debcl | Gain of function | Apoptosis | No effect |
| UAS-P53 H159N | Loss of function | Apoptosis | No effect |
| hop[2]/+ | Loss of function | JAK/STAT | No effect |
| | amorphic | signaling | |
| Stat92E[06346]/+ | Loss of function | JAK/STAT | No effect |
| | amorphic | signaling | |

Generation of UAS-E6 Transgenic Lines

HPV18-E6WT and HPV18-E6V158A are described in Thomas et al. ((2005) The hScrib/Dlg apico-basal control complex is differentially targeted by HPV-16 and HPV-18 E6 proteins. Oncogene 24: 6222-6230). Each cDNA was re-derived by PCR using oligos: 5′-accggtATGGCGCGCTTTGAGGATC-3′ (SEQ ID NO:1) for the 5 prime side of both clones and either 5′-ctcgagTTATACTTGTGTTTCTCTGC-3′ (SEQ ID NO:2) for the wild type 3 prime end, or 5′-ctcgagTTATAGTTGTGTTTCTCTGC-3′ (SEQ ID NO:3) for the Val-to-Ala mutant 3′ end. The underlined codons correspond to the mutated amino acid. All products were subcloned into pGEM-T (Promega) and verified by sequence analysis. Each product was subcloned into pBluescript(KS+) (Stratagene) using Not I and Sac II restriction sites, then transferred into pUASTattB (Bischof J, Maeda R K, Hediger M, Karch F, Basler K (2007) An optimized transgenesis system for Drosophila using germ-line-specific phiC31 integrases. Proc Natl Acad Sci USA 104: 3312-3317) modified to contain either Myc, HA, or Flag epitope tags 5′ to the multiple cloning site, using Age I and Xho I restriction sites. Injections and integrase-mediated insertion into w-; P(CaryP)attP2 was carried out by Rainbow Transgenic Flies, Inc (Camarillo, Calif., USA).

Immunohistochemistry

For immunolabeling, wing discs from wandering third instar larvae and pupal eyes 42 hours after puparium formation were dissected in PBS and fixed in 4% formaldehyde. Fixed tissues were washed three times in PBS solution containing 0.1% Triton-X-100 and blocked in 5% normal goat serum for 1 hour before incubation with primary antibodies. The primary antibodies used in this Example were rabbit anti-Magi 1:200, rabbit anti-Baz 1:1000, mouse anti-Dlg 1:50 (4F3, Developmental Studies Hybridoma Bank), rat anti-DE-cadherin DCAD2 1:50 (Developmental Studies Hybridoma Bank), mouse anti-Flag 1:300 (M2, Sigma), rabbit anti-cleaved Cas3 1:300 (Cell Signaling), rabbit anti-Myc 1:200 (Abcam) and rabbit anti-HA 1:200 (Abcam), rat anti-Magi 1:300, mouse anti-Arm 1:50 (Developmental Studies Hybridoma Bank), mouse anti-MMP1 1:100 (Developmental Studies Hybridoma Bank), rabbit anti-aPKC zeta C20 1:1000 (Santa Cruz Biotechnology). The appropriate secondary antibodies were conjugated Alexa488, Alexa594, and Alexa 647 (Invitrogen).

Images were collected using a Leica SP8 Scanning multiphoton confocal microscope, processed in ImageJ. Figures were assembled using Adobe Photoshop. Low magnification images were collected on a Zeiss Axioskop with a 5× NA 0.50 air lens and AxioVision software. For quantification of protein levels, the fluorescence intensity at the plasma membrane was measured for a standardized ROI in both the overexpression and non-overexpression sides, using the “Find Edges” function of ImageJ followed by threshold adjustments and measurement of intensity. The data were then transferred to Excel for further analysis and plot creation. For each experiment five wing imaginal discs were analyzed and an unpaired t-test was used for statistical analysis.

For quantification of the degree of rescue by Magi::cherry, MagiΔWW, MagiΔPDZ, Dlg::GFP, Scrib::GFP, flies that fulfilled all the following criteria were considered rescued: 1. full wings, 2. only small melanized blisters, 3. fully differentiated wing veins and margins. Flies with rescued wing phenotypes were counted and divided by the total number of flies assayed and percentage was calculated.

Scanning Electron Microscope of Fly Eyes

For SEM of fly eyes heads were fixed in 4% glutaraldehyde in PBS, washed three times to remove the fixative and subsequently dehydrated in an ethanol series. After dehydration samples were critical point dried and mounted on SEM stubs followed by sputter coating with a thin layer of AuPd. Samples were imaged using a Zeiss Neon high-resolution scanning electron microscope.

In Vitro Degradation Assays

These assays were performed as described previously in Glaunsinger B A, Lee S S, Thomas M, Banks L, Javier R (2000) Interactions of the PDZ-protein MAGI-1 with adenovirus E4-ORF1 and high-risk papillomavirus E6 oncoproteins. Oncogene 19: 5270-5280. Briefly, Drosophila Magi and mammalian MAGI-1 proteins were transcribed and translated in vitro, using the TnT kit (Promega), and radiolabelled with [35S]-Methionine (GE Healthcare). They were incubated at 30° C. for the indicated times, with or without the addition of similarly translated HPV-16 and HPV-18 E6 proteins. The remaining proteins were detected by SDS-PAGE and autoradiography.

### Results

Establishing a Fly Model of HPV E6 and Human UBE3A/E6AP

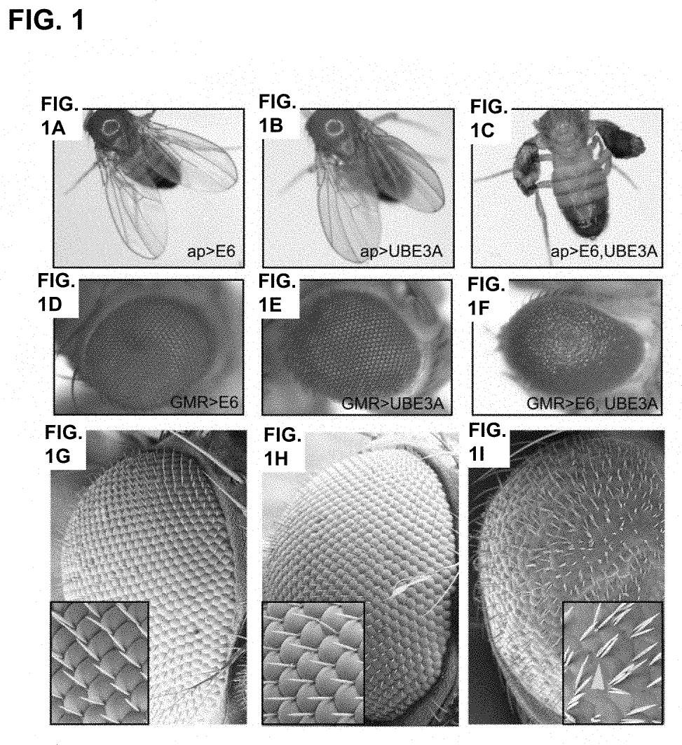

While transgenic mouse models of HPV E6 have significantly contributed to the understanding of HPV-mediated tumorigenesis, the role of the HPV-E6 PDZ targets in vivo has not been clearly established. As there are no other in vivo models of HPV E6 at present, and given the wide array of genetic tools and techniques available in fruit flies, developing a fly E6 model would provide the ability to further dissect the molecular pathways and identify novel partners involved in HPV E6-mediated cellular transformation. To do this, genes encoding the human HPV18 E6 tagged with Myc were expressed in two separate epithelia using the Gal4-UAS high-expression system. When E6 was expressed in the wing or eye epithelia, using the apterous-GAL4 and GMR-GAL4, respectively, no abnormalities within these tissues were detected (FIGS. 1A, 1B, 1D, 1E, 1G, and 1H). E6 requires the human E3 ubiquitin ligase (UBE3A/E6AP) for many of its functions. While Drosophila has a UBE3A homologue, it was possible that HPV E6 was unable to activate or interact with the Drosophila UBE3A. This indicates that the human UBE3A has a specific interaction with E6 or a function that is not conserved in the Drosophila UBE3A protein. The former is likely as the LXXLL motif necessary for E6 binding to human UBE3A is absent from Drosophila UBE3A. In support of this, it was found that expression of E6 together with a previously established transgene that expresses human UBE3A resulted in severe abnormalities in wing and eye epithelia (FIGS. 1C, 1F, and 1I). These abnormalities included smaller and blistered wings that were full of melanized tissue and were held out. Similarly, co-expression of E6 and human UBE3A in the eye resulted in rough eyes with disorganized and fused ommatidia as well as an increase in the number of bristles (FIGS. 1F and 1I). These abnormalities were specific to co-expression of E6 and UBE3A and were 100% penetrant, whilst neither E6 nor UBE3A expression alone resulted in any defects. These results show that expression of E6+UBE3A is deleterious in Drosophila epithelia, suggesting a conservation of cellular targets downstream of the E6+UBE3A complex.

E6 Degrades Drosophila PDZ Domain Proteins

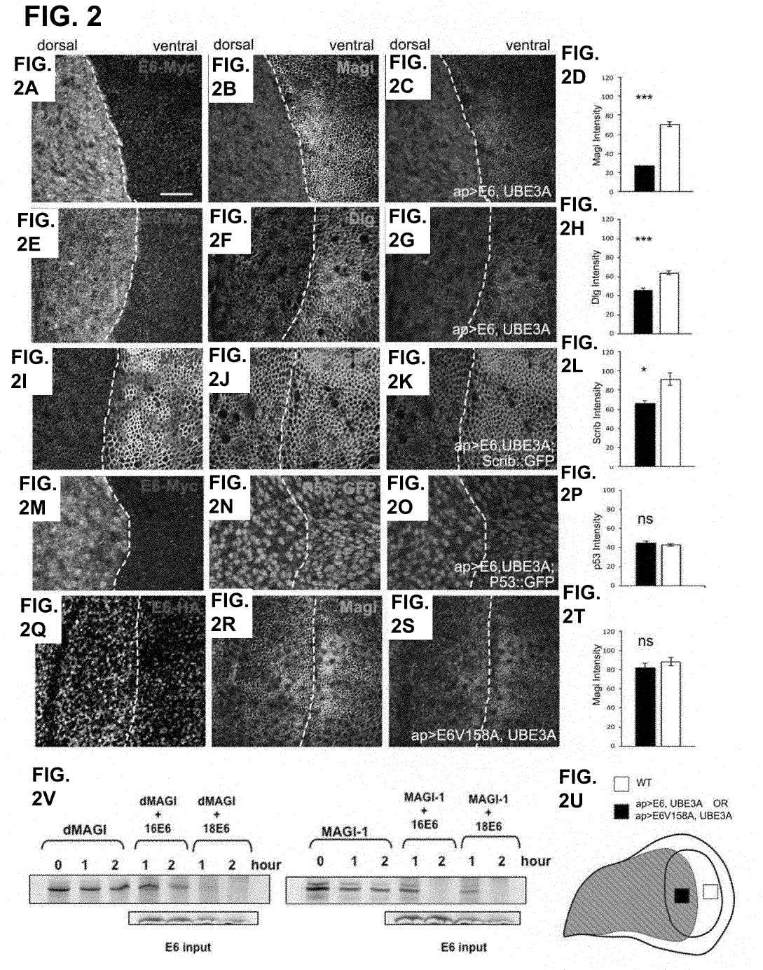

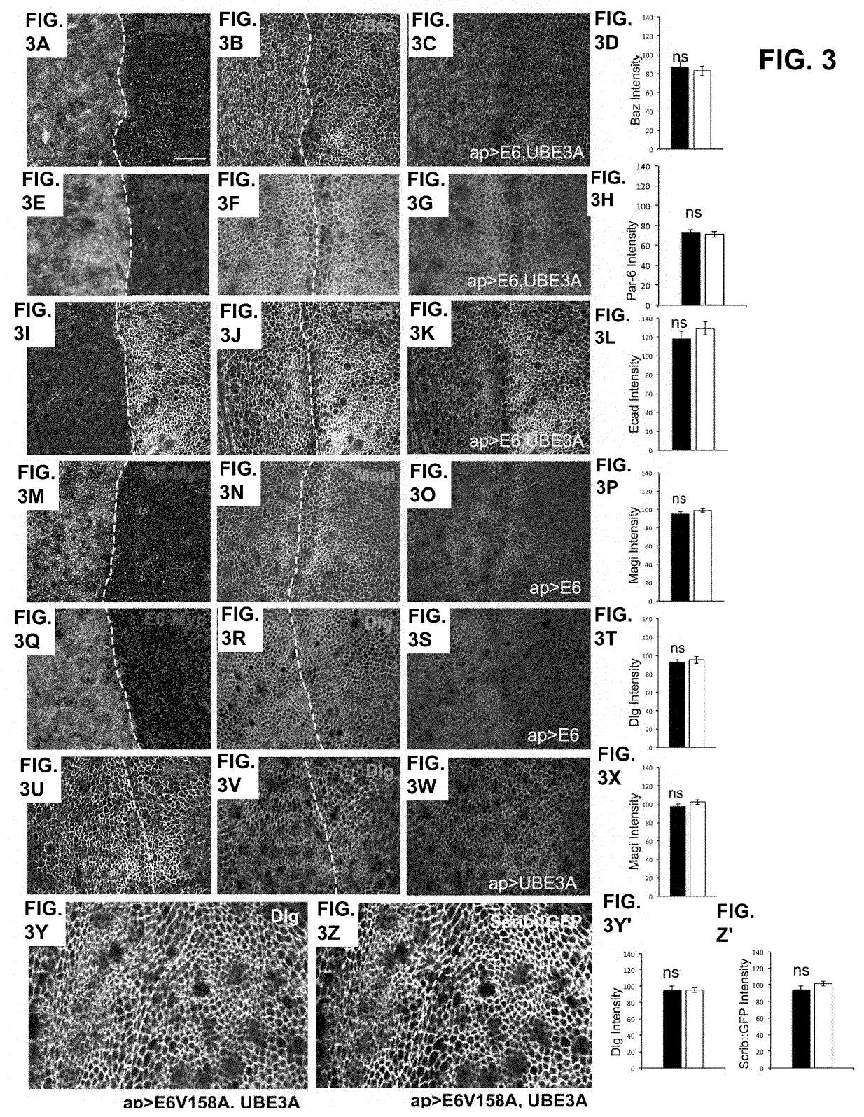

The wing and eye phenotypes prompted the question of whether any of the known targets of E6 in human cells are also targeted in Drosophila epithelia. E6 targets PDZ domain proteins of the polarity and junctional network including MAGI-1, hDlg1 and Scribble/Vartul for ubiquitin-mediated proteasomal degradation, an activity that requires an intact PBM at the extreme C terminus of E6. The wing imaginal disc using the apterous-GAL4 driver to drive expression in the dorsal half of the wing disc was focused on to allow for direct comparison of protein levels in the presence and absence of E6+UBE3A. Further, the Drosophila homologues of Magi, Discs-large (Dlg) and Scribble (Scrib) were focused on and the protein levels on the dorsal (expressing E6+UBE3A) versus ventral (lacking E6+UBE3A) side of the wing imaginal disc were monitored (FIGS. 2A-2L). It was found that E6+UBE3A co-expression led to a significant loss of Magi from the adherens junction domain (FIGS. 2A-2D), a modest reduction in the levels of Dlg (FIGS. 2E-2H), and a weak reduction in the levels of Scrib (FIGS. 2I-2L) at their respective locations in the septate junction. In order to determine if all potential PDZ proteins were effectively targeted by E6, the levels and localization of two other PDZ proteins that are known to be in cell junctions, Bazooka (Par-3) or Par-6, were analyzed and no effect on the levels or the localization of these PDZ protein (FIGS. 3A-3H) were found. These results indicate that E6 binding and degradation of a select group of PDZ proteins is specific. Similarly, there were no changes in the level or localization of E-cadherin at the adherens junction (FIGS. 3I-3L). Expression of E6 or UBE3A alone did not result in any changes in the levels or localization of Magi, Dlg or Scribble (FIGS. 3M-3X), confirming that alterations to the PDZ proteins are a result of the E6 and UBE3A complex. These results are consistent with previous results from human cervical cancer cells in which MAGI-1, hDlg-1 and hScrib are targeted for degradation by E6. In contrast, Drosophila p53 was not targeted for degradation by E6+UBE3A (FIGS. 2M-2P), which is consistent with evolutionary differences between human and fly p53. The results suggest that Drosophila Magi is particularly susceptible to E6 targeting. This is consistent with studies performed in mammalian cells, where MAGI-1 is one of the most strongly bound E6 PDZ targets and is also very efficiently degraded. This suggests that the mechanism of E6 targeting these PDZ cell polarity-regulating proteins is conserved between humans and Drosophila.

As Magi appears to be a major target of HPV E6+UBE3A in Drosophila, there was an interest in directly comparing the ability of E6 to degrade human MAGI-1 and Drosophila Magi in vitro (FIG. 2V). Drosophila Magi and human MAGI-1 were transcribed and translated in vitro, using the TnT rabbit reticulocyte lysate system, which includes functional UBE3A/E6AP. These proteins were then incubated with similarly translated HPV-16 or HPV-18 E6 and the remaining protein analyzed by SDS PAGE and autoradiography. As can be seen from FIG. 2V, Drosophila Magi and mammalian MAGI-1 are almost equivalently susceptible to degradation induced by the high-risk cancer-causing HPV 16 and 18 E6 proteins. These findings highlight the high degree of evolutionary conservation in this particular HPV E6 target, and provide the molecular basis for the results obtained in vivo.

Degradation of PDZ Proteins Requires the E6 PBM

HPV E6-mediated degradation of PDZ proteins in human epithelial cells requires an intact PBM at the C-terminus of the E6 protein. It was next determined whether the loss or reduction of PDZ proteins in the Drosophila epithelial cells was also dependent upon an intact E6 PBM. To do this, a transgene expressing E6 with a point mutation that disrupts the E6 PBM (E6V158A) and prevents E6 binding to PDZ domain proteins was generated. When co-expressed with UBE3A, the E6V158A mutant did not trigger a loss of Magi (FIGS. 2Q-2T), Dlg or Scrib (FIGS. 3Y-3Z′). These results confirm that E6 targeting of Drosophila Magi requires an intact E6 PBM.

Co-Expression of HPV E6 and E6AP Causes Cellular Abnormalities in the Eye and Wing Epithelia

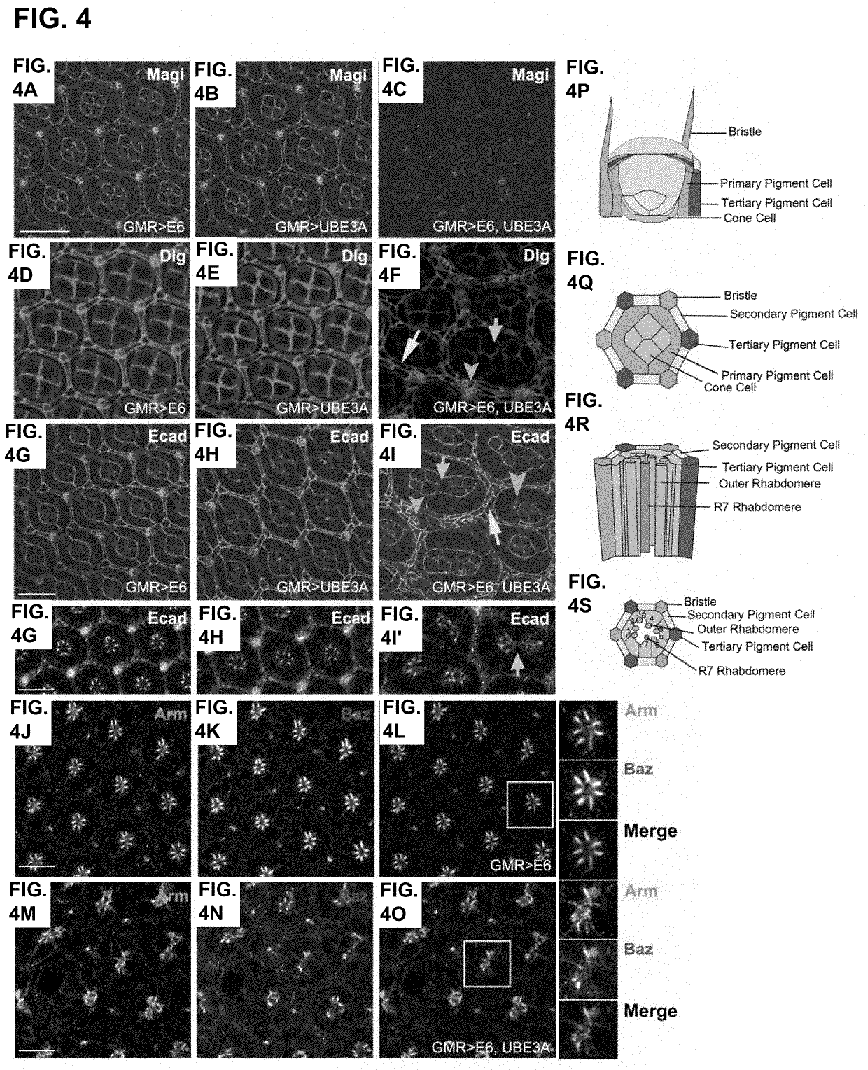

The columnar epithelia of the Drosophila imaginal disc are ideal for studying cellular transformation and cellular signaling pathways, and have been used as in vivo models for detailed analysis of cellular and molecular mechanisms underlying cell polarity defects and cancers. The eye disc in particular has been an excellent model to study the molecular and signaling mechanisms that underlie cell transformation and progression to cancer. When E6+UBE3A was co-expressed in the eye, the earliest cellular phenotypes were observed during pupal stages of development. A loss of Magi (FIG. 4C) and a reduction in Dlg in the eye imaginal disc was observed in the presence of E6+UB3EA (FIG. 4F): this mirrors what was observed in the wing imaginal disc where Magi levels were extensively reduced while Dlg was less so. E6+UBE3A co-expression in the eye resulted in severe tissue abnormalities. In the normal eye, ommatidia are arranged in a hexagonal array to produce a stereotyped pattern. Each ommatidium consists of eight photoreceptor cells, four cone cells, three types of pigment cells (primary, secondary and tertiary) and bristle cells that form the bristles in the adult eye (FIG. 4P). In eyes co-expressing E6 and UBE3A, the overall structure of the eye and the organization of ommatidia was disrupted. Frequently, neighboring ommatidia were fused (70% of ommatidia; n=10 discs) and there was an increase in the number of cone cells, primary, secondary and tertiary pigment cells, as well as bristle cells (FIGS. 4F and 4I). These cellular defects were evident in the adult eye with an increase in the number of bristles and the rough or glossy eye phenotype (FIGS. 1G-1I). Immunolabeling for junctional and polarity markers, Ecad, beta-catenin (Drosophila Armadillo, Arm) and Par3 (Drosophila Bazooka, Baz), revealed that the organization of photoreceptor cells within each ommatiduim was perturbed and that junctional as well as polarity proteins were mislocalized (FIGS. 4G-4O). The displacement of these markers suggests that E6 disrupts the cell polarity and junctional integrity of photoreceptors in ommatidia.

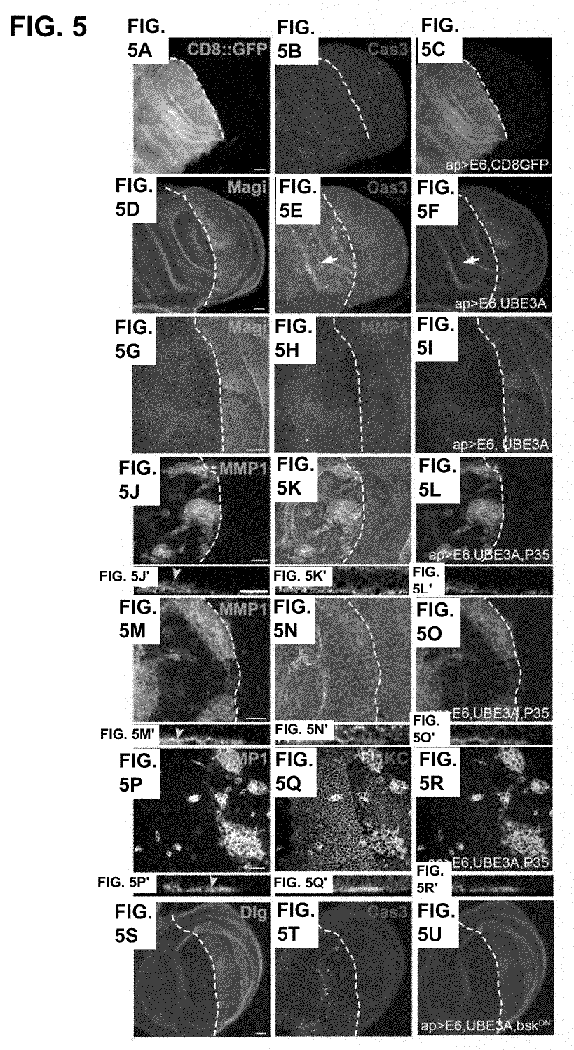

To determine whether the effects of E6+UBE3A were dosage dependent, the expression of E6 and UBE3A were increased by increasing the temperature to 29° C. to increase the efficacy of Gal4. At 29° C., E6+UBE3A triggered extensive apoptosis on the apterous side compared with the control wildtype side, as detected by an antibody to cleaved-Caspase 3 (FIGS. 5D-5F). Normally, expression of the baculovirus protein p35 blocks apoptosis, leading to compensatory proliferation or apoptosis-induced proliferation. Blocking apoptosis with p35 did not result in overgrowth when co-expressed with E6+UBE3A, but instead resulted in clusters of cells that expressed high levels of the polarity proteins, Baz and aPKC (FIGS. 5J-5L) in all discs observed (n=20). Increased expression of vertebrate Par3 (Baz) and aPKC are associated with tumorigenesis and progressive stages of cancer and EMT. A common marker of transformation and EMT is the increased expression of matrix metalloproteinase 1 (MMP1) and it was observed that all cell clusters expressed high levels of MMP1 (FIGS. 5J-5R), suggesting that these cells were undergoing EMT. The cell clusters were not within the columnar epithelia, but were found under the epithelium at the basal side (FIGS. 5J′-5R′; arrowheads). It was also observed that individual cells expressed high levels of MMP1 and polarity proteins away from the cell clusters within the basal side (FIGS. 5P-5R, arrow). Conversely, when the level and localization of the adherens junction protein Ecad was examined, no increase in the delaminated cell cluster was detected. These results suggest that HPV E6, in cooperation with UBE3A, triggers apoptosis and subsequent cell delamination when apoptosis is blocked.

Consistent with previous studies from vertebrates, E6+UBE3A expression alone was insufficient to induce cellular transformation and only when it is paired with processes that block apoptosis is cell transformation observed. As c-Jun N-terminal kinase (JNK) is one of the main signaling pathways triggering apoptosis and MMP1 expression, a dominant negative form of Drosophila JNK (bsk^{DN}) was expressed to block JNK signaling in wing discs co-expressing E6+UBE3A. Blocking JNK signaling did not suppress the E6+UBE3A-mediated cell death (FIGS. 5S-5U), suggesting that the E6+UBE3A mediated apoptosis did not involve the JNK signaling pathway and was driven through another cellular pathway.

Overexpression of dMagi Rescues the Abnormalities Caused by HPV E6+UBE3A

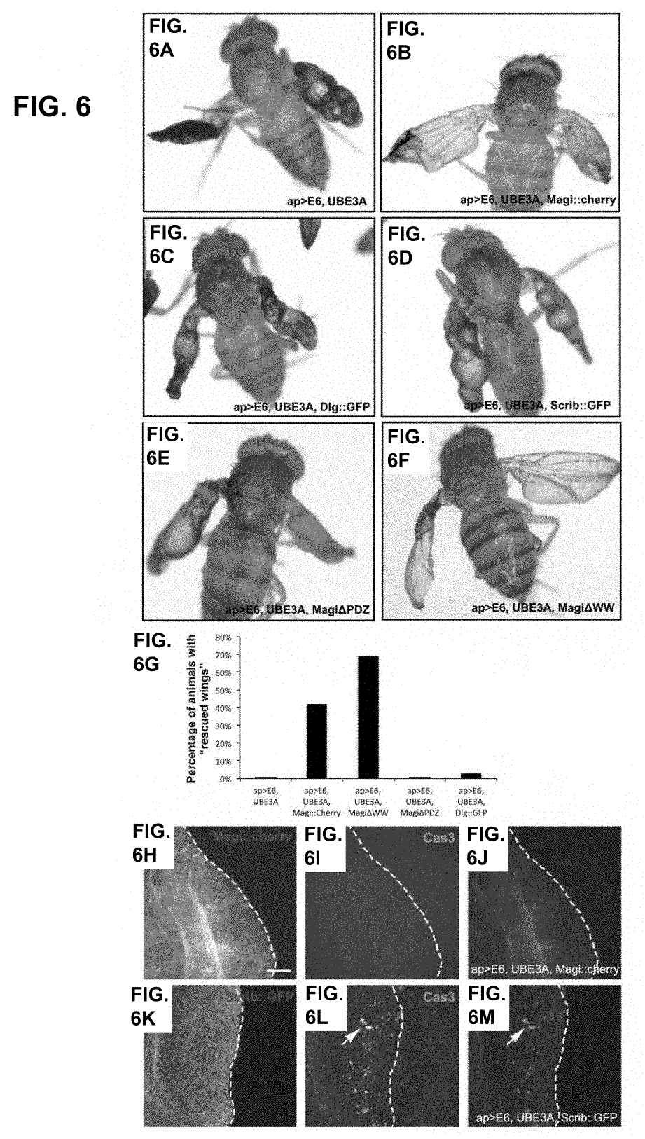

As Magi was strongly reduced by E6+UBE3A expression, it was next analyzed whether Magi overexpression could suppress and rescue the defects caused by E6+UBE3A in the wing epithelia. It was found that co-overexpression of a Cherry-tagged form of Drosophila Magi (Magi::Cherry) and E6+UBE3A partially rescued the adult wing phenotypes (FIG. 6B). Specifically, there was less melanized tissue, a reduced degree of blistering and the flies exhibited full wings with fully differentiated veins and wing margins. The degree of rescue of the wing phenotypes was quantified and there was found a significant reduction in aberrant wing phenotypes when Magi was co-expressed with E6+UBE3A (FIG. 6G). Consistent with this result, E6+UBE3A expressed in wing discs in a null mutant of Magi (Magi^{bst}) resulted in pupal lethality, suggesting an enhancement of the phenotype. To determine whether Magi-mediated rescue of E6+UBE3A was specific, the rescue capability of other E6 PDZ target proteins, including Dlg and Scrib were examined; neither Dlg nor Scrib could suppress the effects of E6+UBE3A (FIGS. 6C, 6D and 6G). Examination of wing discs of third instar larvae revealed that overexpression of Magi completely blocked the apoptosis seen in E6+UBE3A co-expressing epithelia (n=20 discs), whereas overexpression of Scrib did not (n=10 discs) (FIGS. 6H-6M). These results suggest that Magi-mediated rescue of wing abnormalities is specific and that expression of Magi can block or reduce the defects caused by E6+UBE3A co-expression.

Next, it was determined which domains of Magi were responsible for suppressing the E6+UBE3A mediated defects. Drosophila Magi contains four PDZ domains and two WW domains. Magi transgenes lacking either the two WW domains (MagiΔWW) or the PDZ domains (MagiΔPDZ) were expressed in the wing disc, along with E6+UBE3A. Expression of MagiΔWW significantly rescued the wing abnormalities caused by E6+UBE3A (FIGS. 6F & 6G). Conversely, expression of MagiΔPDZ failed to rescue the E6+UBE3A-mediated wing defects (FIGS. 6E & 6G). These results indicate an essential role for the PDZ domains of Magi in blocking the deleterious effects of E6+UBE3A. While Magi is unlikely to be the sole PDZ protein degraded by E6+UBE3A, the data suggests that Magi is an important degradation target and that the highly conserved PDZ domains play a critical role in targeting by HPV E6 and human UBE3A.

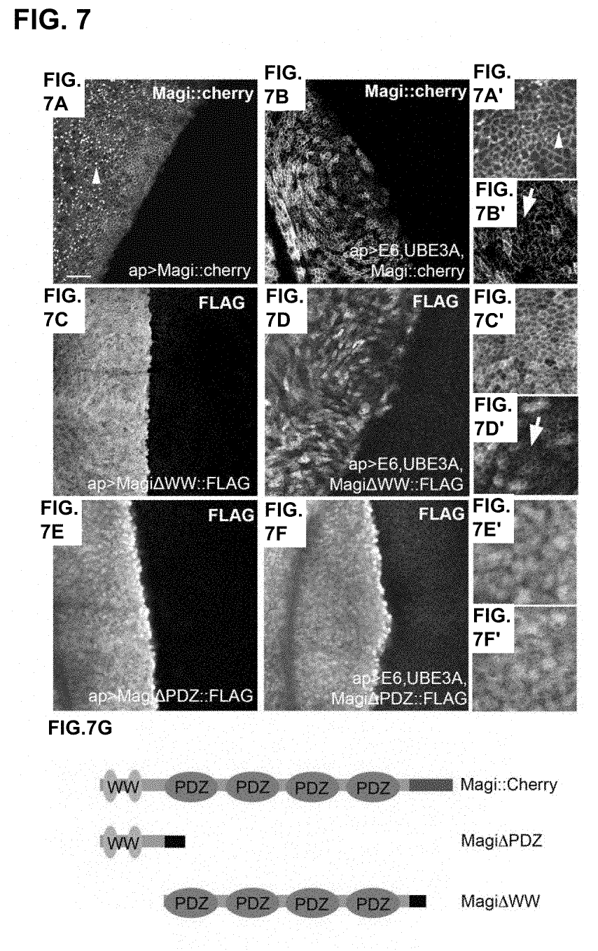

As neither Magi::Cherry nor MagiΔWW were able to fully rescue the defects caused by E6+UBE3A, the degree to which each could be targeted for degradation by E6 was tested. The levels and localization of Magi::Cherry was examined as well as the MagiΔWW and MagiΔPDZ mutants in wing imaginal discs co-expressing E6+UBE3A. Using apterous-GAL4 to drive expression in the dorsal half of the disc, both Magi::Cherry and MagiΔWW proteins were reduced at the plasma membrane although this reduction was not uniform (FIGS. 7B & 7D). The Magi::Cherry expressed alone is uniformly distributed around the membrane and found in prominent intracellular puncta (FIG. 7A), whilst in the presence of E6+UBE3A Magi::Cherry levels are reduced in the puncta and at the membrane. Conversely the localization and expression level of MagiΔPDZ was not affected when co-expressed with E6+UBE3A (FIGS. 7E &7F). These results demonstrate that E6 was capable of degrading a proportion of the wild type Magi::Cherry and MagiΔWW proteins even when these were overexpressed, and this is the most likely the reason why a full rescue of the wing abnormalities was not obtained.

HPV E6 in Conjunction with Oncogenic Ras and Notch Causes Tumorigenesis and Malignancy

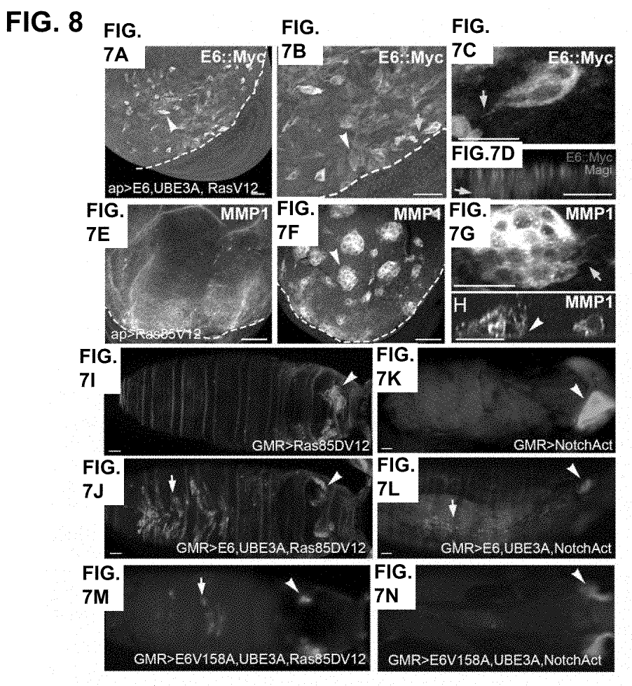

In humans there is usually a period of 15-20 years from the time of HPV infection to the development of cancer. Transgenic mice also showed a latency of 16-20 months for cancer development in presence of HPV E6. These results suggest that cooperation between E6 and UBE3A is insufficient to cause uncontrolled growth and malignant transformation of epithelia, and that genetic events, such as genomic instability and spontaneous mutation, may also play a role in E6-induced epithelial transformation. These results are consistent with this view, as co-expression of E6+UBE3A was not sufficient to cause cellular transformation and cancer. Mutations in oncogenes such as Ras have been implicated in cancer progression and HPV-mediated tumorigenesis. To investigate this in the present model system, E6+UBE3A was expressed in epithelial cells that expressed an activated Ras, Ras85DV12. As expression of Ras85DV12 driven by apterous-Gal4 is larval lethal, a temperature shift experiment was carried out where Gal4 was silenced during embryonic development using a temperature-sensitive Gal4 inhibitor, Gal80^{ts}. A shift to 29° C. during the second instar larval stage activated Gal4, and the wing imaginal discs of third instar larva after 24 hours at 29° C. showed epithelial cells that expressed high levels of E6 and had altered morphology. These cells' morphologically resembled mesenchymal cells (FIGS. 8A & 8B), in that they acquired a fibroblast-like, flat morphology (FIG. 8B), they displayed filopodial-like processes (FIG. 8C; arrow), and were delaminated at the basal side of the columnar epithelia (FIG. 8D; arrow). However, only a subset of cells on the apterous side expressed high levels of E6::myc (FIG. 8B). As the effects of E6 were found to be dosage-dependent, the timing of expression of E6+UBE3A was increased to 48 hours by shifting to 29° C. during the first instar larval stage. Wing imaginal discs of third instar larva after 48 hours of expression had clusters and individual cells that expressed high levels of MMP1 (100% penetrant; n=20 discs) (FIG. 8F, arrow). Cell clusters also had extending filopodial-like processes (FIG. 8G; arrow) Similar to when E6+UBE3A was co-expressed with p35 (FIGS. 5J′, 5M′ and 5P′), these clusters were found under the epithelium at the basal side (FIG. 8H, arrow) and were limited to the apterous side of the disc with no spread into the wildtype, ventral compartment of the wing disc. Overexpression of Ras85DV12 in the absence of E6+UBE3A caused disc overgrowth as expected, but no mesenchymal-like cells or clusters expressing MMP1 were detected (FIG. 8E). These results indicate that the combination of HPV E6 and UBE3A is insufficient to cause cellular transformation, which requires the additional cooperation of cellular oncogenes, such as activated Ras. It is also clear from this analysis that only a small proportion of E6-expressing cells underwent transformation, as initially only single cells with altered morphologies were observed, which, over time, expanded into clusters in a manner analogous to the clonal development of HPV-induced malignancies.

As HPV E6 has been implicated in the later stages of tumorigenesis, it was then determined whether co-expression of E6+UBE3A and Ras85DV12 could cause malignancy and metastasis. In order to do this, E6+UBE3A, Ras85DV12 and a membrane-bound GFP marker (mCD8::GFP) was expressed using GMR-Gal4 in the eye, so that the migration of malignant cells into other regions of the body was readily detected. Expression of E6+UBE3A in the presence of Ras85DV12 resulted in cellular migration, with many GFP-positive cells detected in the abdomen of these animals (FIG. 8J, arrow), distant from the source of E6+UBE3A+Ras85DV12 expression in the eye imaginal disc (FIG. 8J, arrowhead) (70% penetrant, n=20 animals). GFP-positive cells were not detected in the abdomen when Ras85DV12 was expressed alone (FIG. 8I) (n=10 animals). Expression of Ras 85DV12 alone or in combination with E6 and UBE3A caused pupal lethality with extensive tissue necrosis making the analysis of metastasis in the adult impossible. Similar results were obtained when E6+UBE3A were co-expressed with an activated form of Notch (NotchACT) where E6+UBE3A+NotchACT was lethal at the pharate adult stage (prior to hatching). NotchACT plus E6+UBE3A cells labeled with GFP were observed in the abdomen (FIG. 8L, arrow) distant from the eye imaginal disc (FIG. 8L, arrowhead) (40% penetrant, n=30 animals). GFP positive cells were never observed in the abdomen when NotchACT was expressed alone (FIG. 8K) (n=20 animals). These results indicate that E6+UBE3A, when expressed with activated Ras or Notch, leads to many of the cellular phenotypes associated with EMT and spread of transformed cells throughout the body. In order to test if these phenotypes were dependent on the function of the PDZ binding motif of E6, the E6V158A mutant that is deficient in binding to PDZ proteins was expressed in conjunction with RaS85DV12 and UBE3A. In the absence of the PDZ protein interaction, a reduced level of EMT and cell migration with a penetrance of 50% was observed (n=30 animals) (FIG. 8M). Expression of E6V158A in the presence of activated Notch and UBE3A did not result in cell migration away from the eye disc (100% penetrant, n=20) (FIG. 8N). These results collectively suggest that E6 targeting of PDZ proteins plays a major role in EMT and cell migration induced by E6+UBE3A.

A Signaling Pathway Screen Identified the Insulin Receptor as a Downstream Effector of E6

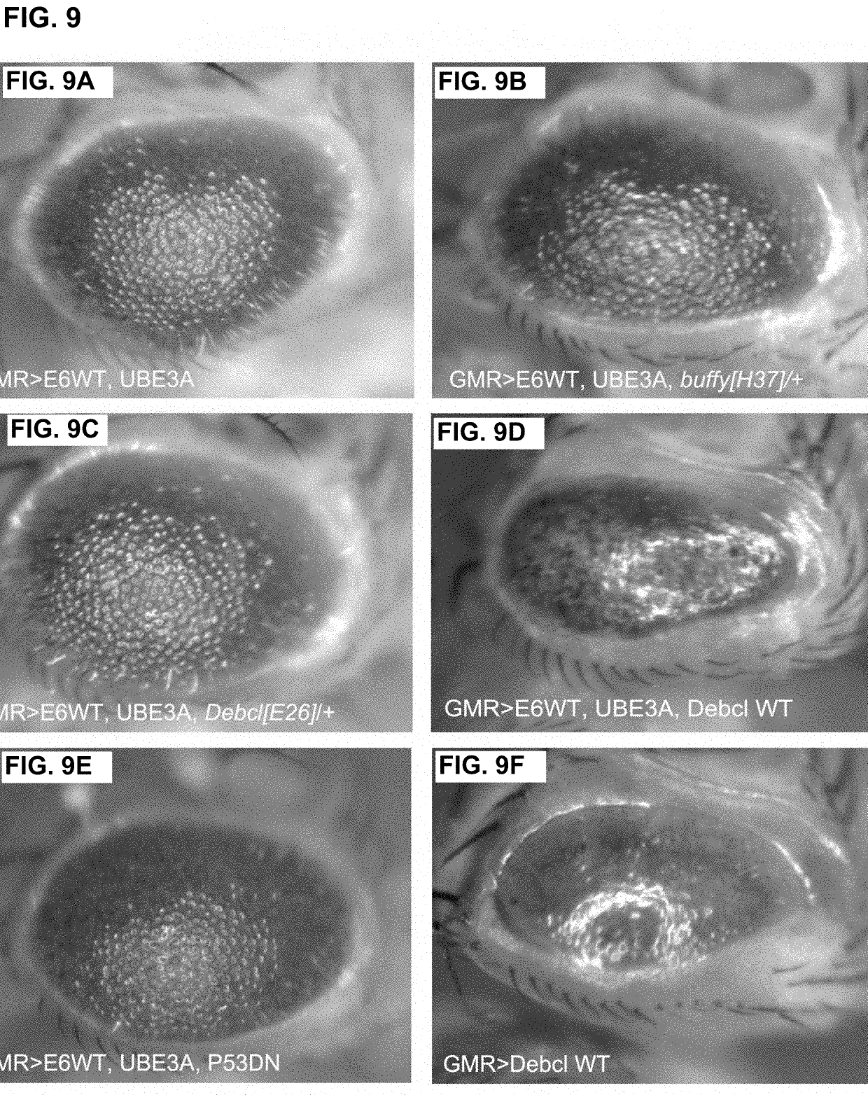

Given the high degree of conservation of its genes and signaling pathways, the Drosophila eye appeared to be an ideal model system to identify the signaling pathways involved in E6+UBE3A-mediated cell abnormalities. Therefore, a small genetic screen was carried out to identify which signaling molecules and pathways functionally interact with E6. A range of signaling pathways (Table 1) were tested and no enhancement or suppression of the E6+UBE3A phenotypes were observed, with the exception of the insulin receptor. Interestingly, no interactions with pathways known to influence apoptosis, such as the JNK pathway, p53 and mutants in the mitochondrial apoptosis pathway (FIG. 9), known to function downstream of the Drosophila retinoblastoma protein (pRb) were found. In particular, blocking p53 function using a DN form did not rescue the eye phenotypes (FIG. 9).

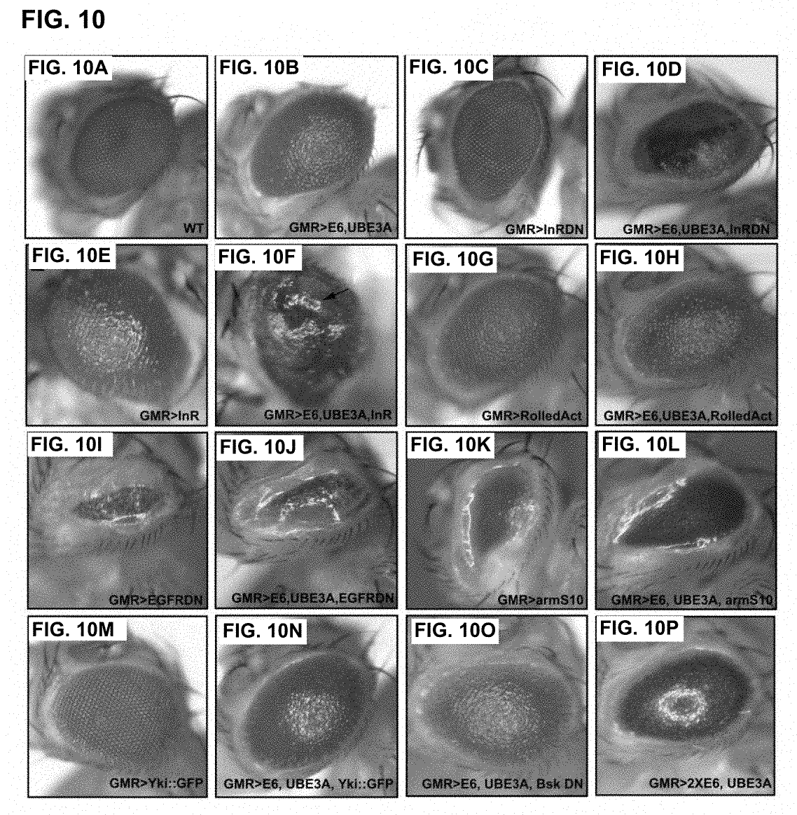

On the other hand, when insulin signaling was blocked by expressing a dominant negative form of the insulin receptor (InRDN) in conjunction with E6+UBE3A, the resulting eyes displayed large necrotic scars (100% of eyes, n=70), which is an indication of cell death (FIG. 10D). Co-expression of the insulin receptor (InR) in E6+UBE3A-expressing eyes resulted in hyperplasia (FIG. 10F; 100% of eyes, n=100) compared with the expression of insulin receptor alone, which generated bigger eyes but no hyperplasia (FIG. 10E). In contrast, changes to the EGF receptor (EGFR) (FIG. 10J), or the MAP kinases ERK (Drosophila Rolled) (FIG. 10H) and JNK (Drosophila Basket) (FIG. 10O), had no effect on the E6+UBE3A eye phenotypes. Similarly, disruption of Wnt signaling with activated beta-catenin (Drosophila Armadillo) (FIG. 10L) or Hippo signaling (Drosophila Yorkie) (FIG. 10N) had no effects. Thus, none of the other pathways tested showed any effect upon the E6+UBE3A phenotype, indicating that the effect of the insulin receptor is specific and suggesting that changes in insulin signaling may play a role in in the cell transformation and cancer progression induced by HPV.

In this model, the levels of E6 expression are high and reflect the higher levels of E6 expression seen during the later stages of malignant progression. It is shown that coexpression of HPV E6 and human UBE3A/E6AP in the wing and eye results in severe morphological defects, whereas E6 or UBE3A expression alone results in none. Further, it is found that E6, in cooperation with UBE3A, targets PDZ proteins in a PBM-dependent manner, and these targets include Magi, Dlg and Scribble with Magi being a major degradation target. In contrast, Drosophila p53 was not degraded by E6+UBE3A. In addition to the loss of PDZ scaffolding proteins, E6+UBE3A expression in epithelia led to apoptosis paired with delamination. Importantly, Magi overexpression rescued the cellular abnormalities caused by E6+UBE3A and the Magi PDZ domains were necessary for this rescue. E6+UBE3A activity was not sufficient to induce tumorigenesis, but did result in malignancy when co-expressed with either an activated form of oncogenic Ras or Notch. These cells had the hallmarks of epithelial-mesenchymal transition (EMT), including morphological changes paired with elevated MMP1 and aPKC expression.

To identify signaling pathways that modulate the E6+UBE3A effects, a genetic screen was conducted, and it was found that E6+UBE3A interacted with the insulin receptor. Overall, the results establish the first Drosophila model to study the HPV E6-mediated cellular transformation and malignancy and suggest a high degree of conservation on the mechanism of HPV E6 mediated cellular transformation.

Various functions and advantages of these and other embodiments of the present disclosure will be more fully understood from the examples shown below. The examples are intended to illustrate the benefits of the present disclosure, but do not exemplify the full scope of the disclosure.

What is claimed is:

1. A transgenic Drosophila co-expressing a gene encoding a human papillomavirus (HPV) E6 oncoprotein and a gene encoding human ubiquitin protein ligase E3A (UBE3A).

2. The transgenic Drosophila of claim 1 further expressing a gene encoding a Drosophila insulin receptor.

3. The transgenic Drosophila of claim 1, wherein co-expression of the gene encoding HPV E6 oncoprotein and the gene encoding human UBE3A is in a tissue selected from the group consisting of eye, wing, and combinations thereof.

4. The transgenic Drosophila of claim 1, wherein the tissue exhibits a cellular abnormality.

5. The transgenic Drosophila of claim 4, wherein the cellular abnormality comprises a small wing size phenotype, a blistered wing phenotype, melanized wing epithelia, rough eye phenotype, glossy eye phenotype, disorganized ommatidia, fused ommatidia, an increase in cone cell number, an increase in primary pigment cell number, an increase in secondary pigment cell number, an increase in tertiary pigment cell number, an increase in bristle cell number, an increase in bristle number, mislocalization of a polarity marker protein, and combinations thereof.

6. The transgenic Drosophila of claim 1, wherein co-expression of the gene encoding HPV E6 oncoprotein and the gene encoding human UBE3A reduces a Drosophila homolog of a human PDZ protein.

7. The transgenic Drosophila of claim 6, wherein the Drosophila homolog of a human PDZ protein is selected from the group consisting of Membrane Associated Guanylate Kinase, WW And PDZ Domain Containing 1 (Magi), discs large homolog 1 scribble cell polarity complex component (Dlg), Scribbled Planar Cell Polarity Protein (Scribble), and combinations thereof.

8. The transgenic Drosophila of claim 1, wherein the HPV E6 oncoprotein comprises a PDZ binding motif.

9. The transgenic Drosophila of claim 1, wherein the HPV E6 oncoprotein is selected from the group consisting of HPV 16 E6, HPV 18 E6, HPV 31 E6, HPV 33 E6, HPV 35 E6, HPV 39 E6, HPV 45 E6, HPV 51 E6, HPV 52 E6, HPV 56 E6, HPV 58 E6, HPV 59 E6, and combinations thereof.

10. A method for screening a candidate therapeutic for Human papillomavirus-induced cell transformation, the method comprising: contacting the candidate therapeutic with a transgenic Drosophila, wherein the transgenic Drosophila co-expresses a gene encoding HPV E6 oncoprotein and a gene encoding human UBE3A;
and analyzing the transgenic Drosophila.

11. The method of claim 10, wherein co-expression of the gene encoding HPV E6 oncoprotein and the gene encoding human UBE3A is in a tissue selected from the group consisting of eye, wing, and combinations thereof.

12. The method of claim 10, wherein co-expression of the gene encoding HPV E6 oncoprotein and the gene encoding human UBE3A results in a cellular abnormality.

13. The method of claim 12, wherein the cellular abnormality comprises a small wing size phenotype, a blistered wing phenotype, melanized wing epithelia, rough eye phenotype, glossy eye phenotype, disorganized ommatidia, fused ommatidia, an increase in cone cell number, an increase in primary pigment cell number, an increase in secondary pigment cell number, an increase in tertiary pigment cell number, an increase in bristle cell number, an increase in bristle number, mislocalization of a polarity marker protein, and combinations thereof.

14. The method of claim 10, wherein co-expression of the gene encoding HPV E6 oncoprotein and the gene encoding human UBE3A reduces a Drosophila homolog of a human PDZ protein.

15. The method of claim 14, wherein the human PDZ protein is selected from the group consisting of Membrane Associated Guanylate Kinase, WW And PDZ Domain Containing 1 (Magi), discs large homolog 1 scribble cell polarity complex component (Dlg), Scribbled Planar Cell Polarity Protein (Scribble), and combinations thereof.

16. The method of claim 10, wherein the transgenic Drosophila further expresses a gene encoding a Drosophila insulin receptor.

17. The method of claim 16, wherein the transgenic Drosophila co-expresses the gene encoding HPV E6 oncoprotein and the gene encoding human UBE3A, and further co-expresses the gene encoding the Drosophila insulin receptor is in a tissue selected from the group consisting of eye, wing, and combinations thereof.

18. The method of claim 17, wherein co-expression of the gene encoding HPV E6 oncoprotein, the gene encoding human UBE3A, and further co-expressing the gene encoding the Drosophila insulin receptor results in excessive overgrowth.

19. The method of claim 10, wherein the HPV E6 oncoprotein is selected from the group consisting of HPV 16 E6, HPV 18 E6, HPV 31 E6, HPV 33 E6, HPV 35 E6, HPV 39 E6, HPV 45 E6, HPV 51 E6, HPV 52 E6, HPV 56 E6, HPV 58 E6, HPV 59 E6, and combinations thereof.

20. A method for screening a candidate therapeutic for Human papillomavirus-induced cancer, the method comprising: contacting the candidate therapeutic with a transgenic Drosophila, wherein the transgenic Drosophila co-expresses a gene encoding HPV E6 oncoprotein and a gene encoding human UBE3A; and analyzing the transgenic Drosophila.


SEQUENCE LISTING (4 sequences)

SEQ ID NO: 1 - DNA, 25 bp, Artificial Sequence
  Synthetic
accggtatggcgcgctttgaggatc

SEQ ID NO: 2 - DNA, 26 bp, Artificial Sequence
  Synthetic
ctcgagttatacttgtgtttctctgc

SEQ ID NO: 3 - DNA, 26 bp, Artificial Sequence
  Synthetic
ctcgagttatagttgtgtttctctgc

SEQ ID NO: 4 - protein, 5 aa, Artificial Sequence
  Synthetic
  misc_feature (2)..(3): Xaa can be any naturally occurring amino acid
LXXLL